SEQUENCE LISTING (4 sequences)

SEQ ID NO: 1 - DNA, 1089 bp, Nicotiana tabacum
  CDS (1)..(1089)
gaagttcgattggatagcttgaaaagctttaatcctcaatcagatatcgatactattatcaaacagtcccctttgcctacccttttcacttacaggcccacttgggaagggggtcagtatgctggtgatgaagtgagtcgactggatgcacttcgagtagcaatggagttgggagctgattacattgatgttgagctaaaggctattgacgagttcaatactgctctacatggaaataaatcagcaaaatgcaaagttattgtttcttctcacaactatgataatacaccatcatctgaggagctcggcaatctagtagcaagaatacaggcatctggagctgacattgtgaagtttgcaacaactgcactggatatcatggatgttgcacgtgtattccaaattactgtacattctcaagtaccaataatagccatggtcatgggagagaagggtttgatgtctcgaatactttgtccaaaatttggtggatacctcacatttggtactcttgaagtgggaaaggtttcggctcctgggcaaccaacaattaaagatcttttgaatatatacaatttcagacagttgggaccagataccagaatatttggcattatcgggaagcctgttagccatagcaaatcacctttattgtataatgaagctttcagatcagttgggtttaatggtgtttatatgcctttgctggttgatgatgttgcaaatttctttcggacttactcatctttagattttgctggctcagctgtaacaattcctcacaaggaagccattgttgactgctgtgatgagttgaatcctaccgctaaagtaataggggctgtcaattgtgtcgtaagccgactcgatgggaagttgtttggttgcaatacagactatgtgggtgcaatctccgccattgaagaagcgttgcaaggctcacagcctagtatgtctgggtctcccttagctggtaaattatttgtggtcattggtgctggtggcgctggcaaggcacttgcttatggtgcaaaggaaaagggggctcgggtggtgattgctaaccgtacctatgaacgagcgagagaa

SEQ ID NO: 2 - protein, 363 aa, Nicotiana tabacum
EVRLDSLKSFNPQSDIDTIIKQSPLPTLFTYRPTWEGGQYAGDEVSRLDALRVAMELGADYIDVELKAIDEFNTALHGNKSAKCKVIVSSHNYDNTPSSEELGNLVARIQASGADIVKFATTALDIMDVARVFQITVHSQVPIIAMVMGEKGLMSRILCPKFGGYLTFGTLEVGKVSAPGQPTIKDLLNIYNFRQLGPDTRIFGIIGKPVSHSKSPLLYNEAFRSVGFNGVYMPLLVDDVANFFRTYSSLDFAGSAVTIPHKEAIVDCCDELNPTAKVIGAVNCVVSRLDGKLFGCNTDYVGAISAIEEALQGSQPSMSGSPLAGKLFVVIGAGGAGKALAYGAKEKGARVVIANRTYERARE

SEQ ID NO: 3 - DNA, 1667 bp, Nicotiana tabacum
  CDS (3)..(1667)
gggaggcaatgacgaggaacgaaacactaatttgtgcaccaatcatggcagacacagtggatcaaatgttgaatctaatgcaaaaggctaaaattagtggtgctgatcttgtggaagttcgattggatagcttgaaaagctttaatcctcaatcagatatcgatactattatcaaacagtcccctttgcctacccttttcacttacaggcccacttgggaagggggtcagtatgctggtgatgaagtgagtcgactggatgcacttcgagtagcaatggagttgggagctgattacattgatgttgagctaaaggctattgacgagttcaatactgctctacatggaaataaatcagcaaaatgcaaagttattgtttcttctcacaactatgataatacaccatcatctgaggagctcggcaatctagtagcaagaatacaggcatctggagctgacattgtgaagtttgcaacaactgcactggatatcatggatgttgcacgtgtattccaaattactgtacattctcaagtaccaataatagccatggtcatgggagagaagggtttgatgtctcgaatactttgtccaaaatttggtggatacctcacatttggtactcttgaagtgggaaaggtttcggctcctgggcaaccaacaattaaagatcttttgaatatatacaatttcagacagttgggaccagataccagaatatttggcattatcgggaagcctgttagccatagcaaatcacctttattgtataatgaagctttcagatcagttgggtttaatggtgtttatatgcctttgctggttgatgatgttgcaaatttctttcggacttactcatctttagattttgctggctcagctgtaacaattcctcacaaggaagccattgttgactgctgtgatgagttgaatcctaccgctaaagtaataggggctgtcaattgtgtcgtaagccgactcgatgggaagttgtttggttgcaatacagactatgtgggtgcaatctccgccattgaagaagcgttgcaaggctcacagcctagtatgtctgggtctcccttagctggtaaattatttgtggtcattggtgctggtggcgctggcaaggcacttgcttatggtgcaaaggaaaagggggctcgggtggtgattgctaaccgtacctatgaacgagcgagagaacttgctgatgtagttggaggtcaggctttgtctcttgacgagcttagcaatttccatccagaaaatgacatgattcttgcaaataccacctccattggcatgcaaccaaaggttgatgatacaccaatctttaaggaagctttgaggtactactcacttgtatttgatgctgtttatacgcccaaaatcactagactcttgcgggaagctcacgagagtggagtaaaaattgtaacaggagttgaaatgtttatcggccaggcatatgaacaatatgagagatttacagggcttgccagctccaaaggaacttttcaagaaaattatggctggatattgagagcaaggtctctttcccttttcaatgcggccctgctagttacttttcctcctaaatccctacatagttgtgtgatagcaatggtcttagattcctctgccctaccatttgtgcttcggaggaat

SEQ ID NO: 4 - protein, 555 aa, Nicotiana tabacum
EAMTRNETLICAPIMADTVDQMLNLMQKAKISGADLVEVRLDSLKSFNPQSDIDTIIKQSPLPTLFTYRPTWEGGQYAGDEVSRLDALRVAMELGADYIDVELKAIDEFNTALHGNKSAKCKVIVSSHNYDNTPSSEELGNLVARIQASGADIVKFATTALDIMDVARVFQITVHSQVPIIAMVMGEKGLMSRILCPKFGGYLTFGTLEVGKVSAPGQPTIKDLLNIYNFRQLGPDTRIFGIIGKPVSHSKSPLLYNEAFRSVGFNGVYMPLLVDDVANFFRTYSSLDFAGSAVTIPHKEAIVDCCDELNPTAKVIGAVNCVVSRLDGKLFGCNTDYVGAISAIEEALQGSQPSMSGSPLAGKLFVVIGAGGAGKALAYGAKEKGARVVIANRTYERARELADVVGGQALSLDELSNFHPENDMILANTTSIGMQPKVDDTPIFKEALRYYSLVFDAVYTPKITRLLREAHESGVKIVTGVEMFIGQAYEQYERFTGLASSKGTFQENYGWILRARSLSLFNAALLVTFPPKSLHSCVIAMVLDSSALPFVLRRN

(19) United States
(12) Patent Application Publication
(10) Pub. No.: US 2003/0145348 A1
(43) Pub. Date: Jul. 31, 2003

(54) Dehydroquinate dehydrase/shikimate dehydrogenase as a herbicide target

(76) Inventors: Annette Freund, Limburgerhof (DE); Uwe Sonnewald, Quedlinburg (DE); Li Ding, Gatersleben (DE)

Correspondence Address: NIXON & VANDERHYE, PC, 1100 N GLEBE ROAD, 8TH FLOOR, ARLINGTON, VA, 22201-4714, US

(21) Appl. No.: 10/332,149

(22) PCT Filed: Jul. 6, 2001
(86) PCT No.: PCT/EP01/07763

(30) Foreign Application Priority Data
Jul. 7, 2000 (DE) 100325939

(51) Int. Cl. C12Q 1/68; G01N 33/53; G01N 33/567; A01N 25/00; A01H 1/00; C12N 15/82

(52) U.S. Cl. 800/278; 435/6; 435/7.2; 504/116.1

(57) ABSTRACT

The present invention relates to the use of nucleic acid sequences encoding a polypeptide with dehydroquinate dehydratase/shikimate dehydrogenase activity for generating an assay system for finding dehydroquinate dehydratase/shikimate dehydrogenase inhibitors. Using antisense techniques, it was shown for the first time that dehydroquinate dehydratase/shikimate dehydrogenase constitutes a target for herbicides. Moreover, the application relates to the generation of transgenic plants comprising a nucleic acid sequence encoding a polypeptide with dehydroquinate dehydratase/shikimate dehydrogenase (E.C. 4.2.1.10/E.C. 1.1.1.25) activity which comprises an increased biomass production and/or an increased content in aromatic amino acids in comparison with a nontransgenic plant.

The present invention relates to the identification of plant dehydroquinate dehydratase/shikimate dehydrogenase (DHD/SHD) as novel target for herbicidally active ingredients. The present invention furthermore relates to a method for generating an assay system based on the use of the DNA sequence SEQ ID No. 1 or SEQ ID No. 3, of functional equivalents of SEQ ID No. 1 or SEQ ID No. 3 or parts of SEQ ID No. 1 or SEQ ID No. 3 encoding a plant polypeptide with dehydroquinate dehydratase/shikimate dehydrogenase activity for identifying inhibitors of plant dehydroquinate dehydratase/shikimate dehydrogenase. The invention also relates to a substance identified using these methods or this assay system and to their use as herbicides or to the use of the polypeptide with dehydroquinate dehydratase/shikimate dehydrogenase activity as target for herbicides.

The present invention furthermore relates to a method for generating transgenic plants comprising SEQ ID No. 1 or SEQ ID No. 3, functional equivalents of SEQ ID No. 1 or SEQ ID No. 3 or parts of SEQ ID No. 1 or SEQ ID No. 3 featuring an increased dry matter and/or an increased aromatic amino acid content in comparison with a nontransgenic plant of the same type.

Furthermore, the invention relates to methods for identifying nucleic acid sequences of dehydroquinate dehydratase/shikimate dehydrogenase variants which are resistant to inhibitors of plant dehydroquinate dehydratase/shikimate dehydrogenase identified by the methods according to the invention, and to transgenic plants which comprise the nucleic acid sequences of said dehydroquinate dehydratase/shikimate dehydrogenase variants.

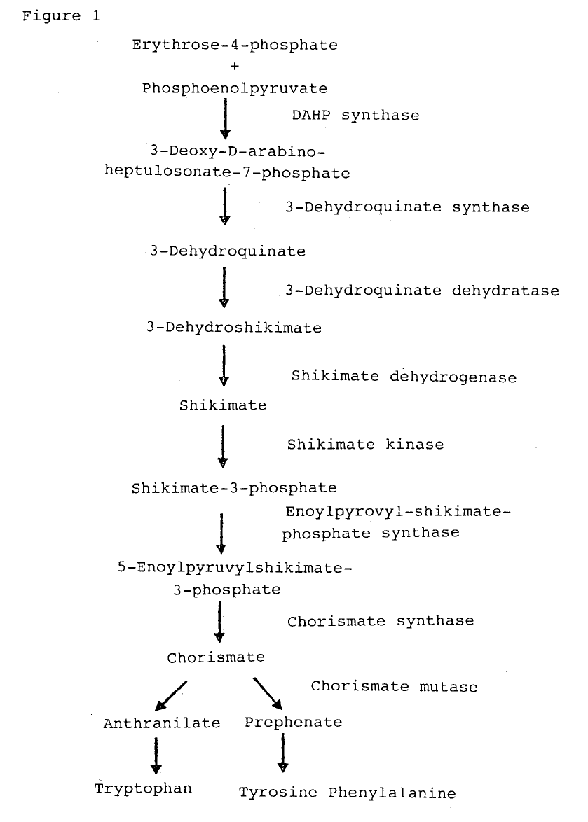

Dehydroquinate dehydratase/shikimate dehydrogenase participates in the biosynthesis of Chorismat, the precursor of the aromatic amino acids phenylalanine, tyrosine and tryptophan, see FIG. 1.

Precursors for the formation of aromatic amino acids are erythrose-4-phosphate and phosphoenolpyruvate. The two substances undergo condensation with elimination of the two phosphates to give 2-keto-3-deoxy-D-arabinoheptulosonate-7-phosphate, a C7 compound which cyclizes to give dehydroquinate. After elimination of water by dehydroquinate dehydratase (E.C. 4.2.1.10) and reduction of the carbonyl group by shikimate dehydrogenase (E.C. 1.1.1.25), shikimate is formed; see Voet and Voet, Biochemie, 1994, Verlag Chemie. Dehydroquinate dehydratase/shikimate dehydrogenase is a bifunctional enzyme which catalyzes the third and the fourth step in Chorismat biosynthesis, see also Mitsuhashi, S., Davis, B. D., Biochim. Biophys. Acta 15, (1954), 54-61; Jacobson, J. W., Hart, B. A., Doy, C. H., Giles, N. H., Biochim. Biophys. Acta 289 (1972) 1-12; Polley, L. D., Biochim. Biophys. Acta 526 (1978) 259-266; Chaudhuri, S., Coggins, J. R. Biochem. J. 226 (1985), 217-223.

A variety of inhibitors have been identified for shikimate dehydrogenase. On the one hand, various metal compounds and metal ions, such as ZnCl₂, CdSO₄, CuSO₄, HgCl₂, Hg²⁺, Zn²⁺, Cu²⁺ and berates have an inhibitory effect on shikimate dehydrogenase (Lourenco, E. J., Neves, V. A., Phytochemistry 23, (1984) 497-499; Lemos Silva, G. M., Lourenco, E. J., Neves, V. A. J. Food Biochem. 9 (1985), 105-116), on the other hand it was demonstrated that arsenites, p-chloromercuribenzoates and N-ethylmaleimides have an inhibitory effect on the enzyme (Sanderson, G. W. Biochem. J., 98 (1966), 248-252). Inhibitors were also identified for dehydroquinate dehydratase. Thus, acetates, succinates, D-(+)-tartrates and diethylcarbonates have an inhibitory effect on dehydroquinate dehydratase in Escherichia coli (Chaudhuri, S., Lambert, J. M., McColl, L. A., Coggins, J. R., Biochem. J., 239, (1986), 699-704; Chaudhuri, S., Duncan, K., Coggins, J. R. Methods Enzymol., 142 (1987), 320-324).

Since plants depend on an efficient amino acid metabolism, it can be assumed that enzymes which participate in amino acid biosynthesis are suitable as target protein for herbicides. Thus, active ingredients have already been described which inhibit plant de novo amino acid biosynthesis. An example which may be mentioned is glyphosate, which inhibits amino acid biosynthesis in planta.

Plant gene sequences for dehydroquinate dehydratase/shikimate dehydrogenase are already known from Glycine max, Gossypium hirsutum, Lycopericum esculentum, Oryza sativa, Nicotiana tabacum and Arabidopsis thaliana.

The shikimate pathway plays a role not only in the biosynthesis of aromatic amino acids, but also in a multiplicity of other substances which are formed in large amounts by the plant, such as, for example, ubiquinone, folate, flavonoids, coumarins, lignin, alkaloids, cyanogenic glucosides, plastoquinone and tocopherols. The total of all of these substances may amount to up to 50% of the dry matter of a plant.

The suitability of an enzyme as a target for herbicides can be confirmed by reducing the enzyme activity, for example by means of antisense technology, in transgenic plants. If the introduction of an antisense DNA for a particular gene into a plant brings about reduced growth, this suggests that the enzyme whose activity is reduced is suitable as the site of action for herbicidal active ingredients. For example, antisense inhibition of acetolactate synthase (ALS) in transgenic potato plants and the treatment of control plants with ALS-inhibiting herbicides lead to comparable phenotypes (Hofgen et al., Plant Physiology 107 (1995), 469-477).

The term transgenic is understood as meaning, for the purposes of the invention, that the nucleic acids used in the method are not located at their natural position in the genome of an organism, it being possible for the nucleic acids to be expressed homologously or heterologously in this context. However, the term transgenic also means that the nucleic acids according to the invention are indeed located at their natural position in the genome of an organism, but that the sequence has been modified in comparison with the natural sequence and/or that the regulatory sequences of the natural sequences have been modified. Preferably, the term transgenic refers to the expression of the nucleic acids at a non-natural position in the genome, that is to say the nucleic acids are expressed homologously or, preferably, heterologously. The same applies to the nucleic acid construct according to the invention or the vector.

It is an object of the present invention to confirm that dehydroquinate dehydratase/shikimate dehydrogenase in plants is a suitable herbicide target, and to generate an effective and simple dehydroquinate dehydratase/shikimate dehydrogenase assay system for carrying out inhibitor-enzyme binding studies. It is a further object to identify dehydroquinate dehydratase/shikimate dehydrogenase variants which are resistant to the inhibitors found in accordance with the invention.

We have found that this object is achieved by isolating DNA sequences which encode the plant enzyme dehydroquinate dehydratase/shikimate dehydrogenase, by the generation of antisense or cosuppression constructs of plant dehydroquinate dehydratase/shikimate dehydrogenase and their expression in plants, and by the functional expression of plant dehydroquinate dehydratase/shikimate dehydrogenase in prokaryotic or eukaryotic cells.

The model plant employed for the expression of dehydroquinate dehydratase/shikimate dehydrogenase in sense and antisense orientation was tobacco. (variety NN Samsun).

To prepare a recombinant enzymne for carrying out enzyme assays, dehydroquinate dehydratase/shikimate dehydrogenase was expressed heterogolously in E. coli.

To achieve the object, a cDNA encoding plant dehydroquinate dehydratase/shikimate dehydrogenase was isolated from tobacco and sequenced, see Example 1 and sequence listings SEQ ID No. 1, SEQ ID No. 3 and Bonner, C. and Jensen, R. Biochem. J. 302 (1994), 11-14. The gene can be overexpressed functionally in various heterologous systems such as in E. Coli, yeasts or baculoviruses and employed in assay systems for identifying inhibitors. Using antisense or cosuppression plants, it has been proven for the first time that dehydroquinate dehydratase/shikimate dehydrogenase constitutes an essential gene for plants.

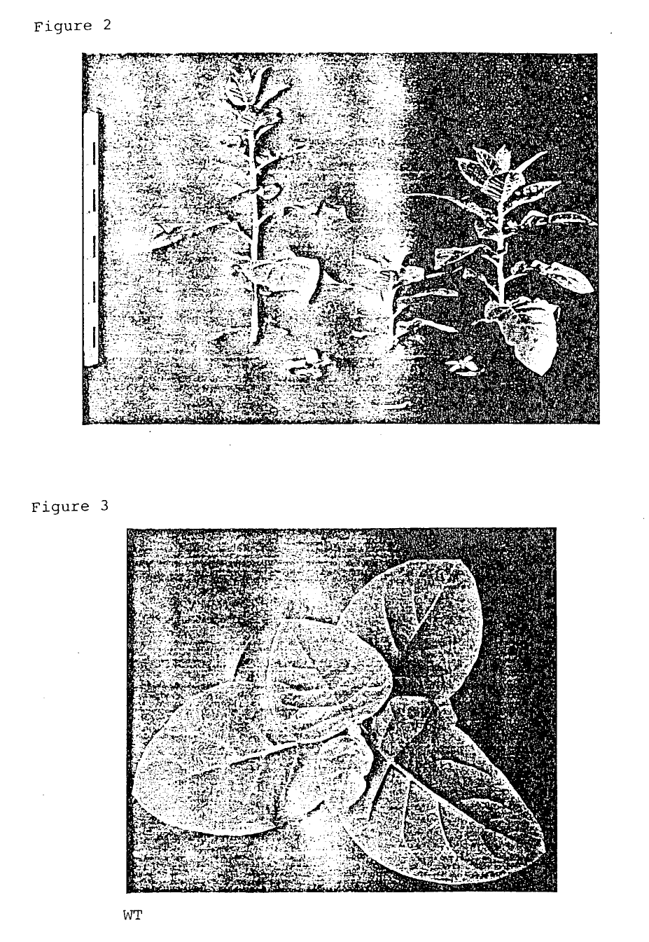

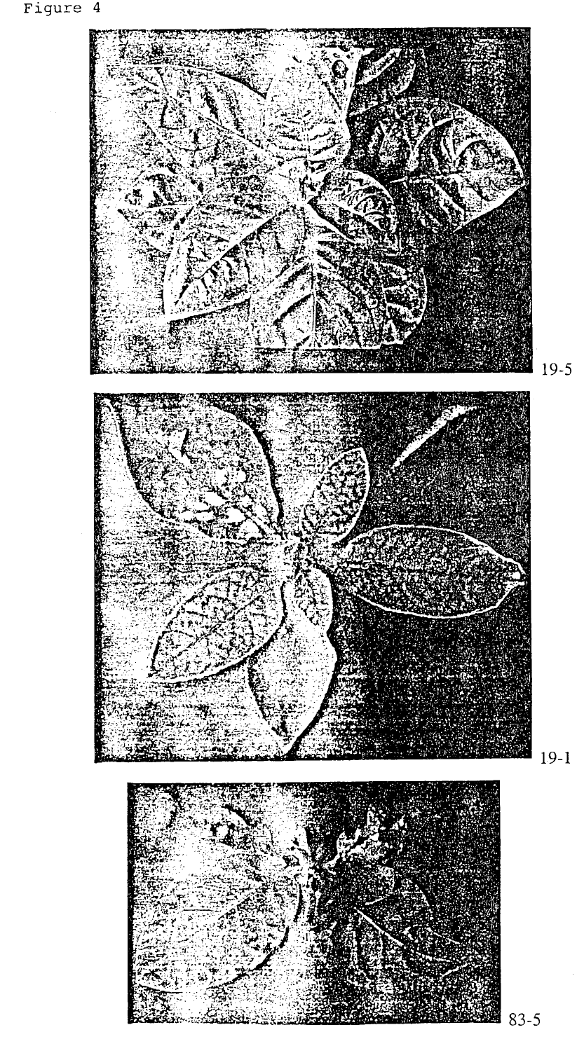

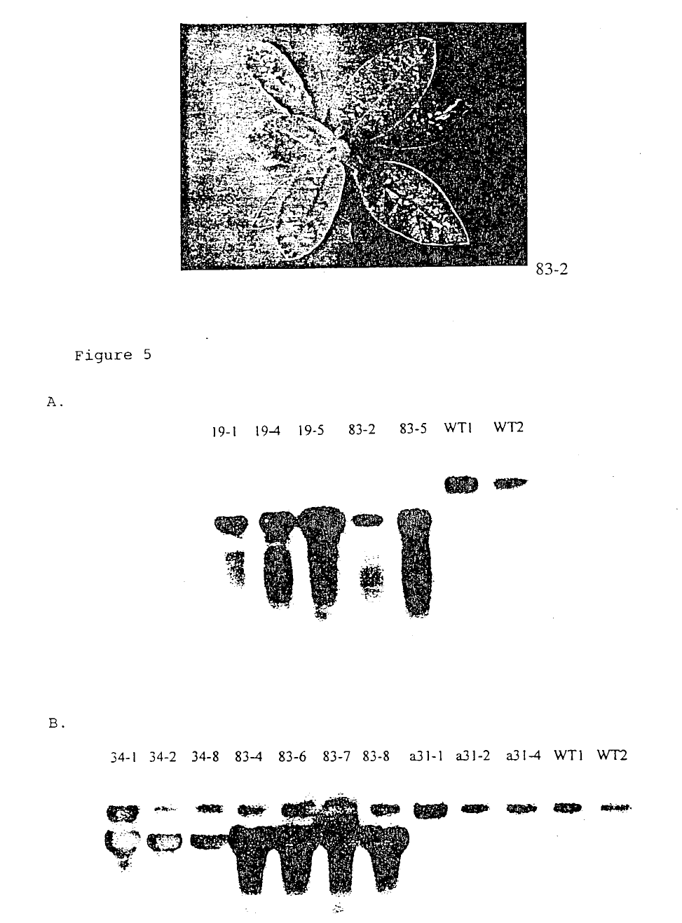

Tobacco plants carrying an antisense construct of dehydroquinate dehydratase/shikimate dehydrogenase—see examples 2 and 3—were characterized in greater detail. The plants showed different degrees of retarded growth. Thus, the wild type and transgenic DHD/SHD plants are shown as a side view (FIG. 2) and from above (FIGS. 3 and 4). It can be seen clearly in transgenic DHD/SHD plants that growth is inhibited greatly compared with the wild type (FIG. 2, wild type outside left). The transgenic lines and the progeny of the 1^{st} and 2^{nd} generations showed reduced growth in soil. In plants with reduced growth, a dehydroquinate dehydratase/shikimate dehydrogenase RNA quantity was detected by Northern hybridization which was reduced compared with that of the wild type, see FIG. 5A. Furthermore, enzyme activity measurement (example 5) detected a reduced amount of dehydroquinate dehydratase/shikimate dehydrogenase activity in transgenic DHD/SHD lines compared with the wild-type plants. The expression level and the reduction of the dehydroquinate dehydratase/shikimate dehydrogenase activity correlate with the level of growth retardation. It has been found that the introduction of a dehydroquinate dehydratase/shikimate dehydrogenase antisense construct results in reduced growth of the plant.

In wild-type tobacco plants and in DHD/SHD cosuppression plants, the activity of the DHD/SHD enzyme was measured by the method as described in example 5. It emerged that, in the case of cosuppression plants, the DHD/SHD enzyme activity is zero, and that an enzyme activity of 0.025-0.06 μM/min/g can be measured in wild-type plants.

This unambiguous relationship identifies dehydroquinate dehydratase/shikimate dehydrogenase for the first time as a suitable target protein for herbicidal active ingredients.

To allow effective inhibitors of plant dehydroquinate dehydratase/shikimate dehydrogenase to be found, it is necessary to provide suitable assay systems with which inhibitor/enzyme binding studies can be carried out.

To generate these assay systems, a nucleic acid sequence for identifying inhibitors of plant dehydroquinate dehydratase/shikimate dehydrogenase can be used, it being possible for said nucleic acid sequence to encompass, for example, the DNA sequence SEQ ID No. 1 or SEQ No. 3 comprising the coding region of a plant dehydroquinate dehydratase/shikimate dehydrogenase, or a nucleic acid sequence which hybridizes with the DNA sequence SEQ No. 1 or SEQ ID No. 3 or parts or derivatives derived from these sequences by insertion, deletion or substitution, and which nucleic acid sequence encodes a protein having the biological activity of a plant dehydroquinate dehydratase/shikimate dehydrogenase.

More accurately, the invention thus furthermore relates to methods for identifying novel herbicides based on the use of a protein with dehydroquinate dehydratase/shikimate dehydrogenase activity encoded by a nucleic acid sequence, which nucleic acid sequence encompasses the following sequence:

a) a nucleic acid sequence with the sequence shown in SEQ ID No. 1 or SEQ ID No. 3; or

b) a nucleic acid sequence which, owing to the degeneracy of the genetic code, can be deduced from the amino acid sequences shown in SEQ ID No. 2 or SEQ ID No. 4 by backtranslation; or

c) functional analogs of the nucleic acid sequences shown in SEQ ID No. 1 or SEQ ID No. 3 which encode a polypeptide with the amino acid sequences shown in SEQ ID No. 2 or SEQ ID No. 4; or

d) functional analogs of the nucleic acid sequence shown in SEQ ID No. 1 or SEQ ID No. 3 which encode functional analogs of the amino acid sequences shown in SEQ ID No. 2 or SEQ ID No. 4; or

e) parts of the nucleic acid sequences a), b), c) or d); or

f) at least 300 nucleotide units of the nucleic acid sequences a), b), c) or d).

It is advantageous in this context to use polypeptides with dehydroquinate dehydratase/shikimate dehydrogenase activity with an amino acid sequence homology with the tobacco dehydroquinate dehydratase/shikimate dehydrogenase with SEQ ID No. 2 or SEQ ID No. 4 of 20-100%, preferably 50-100%, especially preferably 70-100%, very especially preferably 80-100%, or 85-100%, or 90-100%, or 95-100%, or 96-100%, or 97-100%, or 98-100%, or 99-100%.

Homology between two nucleic acid sequences or polypeptide sequences is defined by the identity of the nucleic acid sequence/polypeptide sequence over in each case the entire sequence length, which is calculated by alignment with the aid of the program algorithm GAP (Wisconsin Package Version 10.0, University of Wisconsin, Genetics Computer Group (GCG), Madison, USA), setting the following parameters:

| | |
|---|---|
| Gap Weight: 12 | Length Weight: 4 |
| Average Match: 2.912 | Average Mismatch: −2.003 |

Functional analogs or functionally equivalent sequences which encode a dehydroquinate dehydratase/shikimate dehydrogenase gene are those sequences which, despite a deviating nucleotide sequence, retain the desired function. Thus, functional equivalents encompass naturally occurring variants of the sequences described herein, but also artificial, for example chemically synthesized, artificial nucleotide sequences (50) which are adapted to the codon usage of an organism, but also sequences which hybridize with the sequences according to the invention or parts of these sequences.

To carry out hybridization, it is advantageous to use short oligonucleotides, for example of the conserved or other regions, which can be determined in the manner with which the skilled worker is familiar by comparisons with other related genes. However, longer fragments of the nucleic acids according to the invention, or the complete sequences, may also be used for hybridization. Depending on the nucleic acid/oligonucleotide longer fragment or complete sequence used, or depending on which type of nucleic acid, i.e. DNA or RNA, is being used for the hybridization, these standard conditions vary. Thus, for example, the melting temperatures for DNA:DNA hybrids are approximately 10° C. lower than those of DNA:RNA hybrids of the same length.

Standard hybridization conditions are to be understood as meaning, depending on the nucleic acid, for example temperatures of between 42 and 58° C. in an aqueous buffer solution with a concentration of between 0.1 to 5×SSC (1×SSC=0.15 M NaCl, 15 mM sodium citrate, pH 7.2) or additionally in the presence of 50% formamide, such as, for example, 42° C. in 5×SSC, 50% formamide. The hybridization conditions for DNA:DNA hybrids are advantageously 0.1×SSC and temperatures of between approximately 20° C. to 45° C., preferably between approximately 30° C. to 45° C. In the case of DNA:RNA hybrids, the hybridization conditions are advantageously 0.1×SSC and temperatures of between approximately 30° C. to 55° C., preferably between approximately 45° C. to 55° C. These hybridization temperatures which have been stated are melting temperature values which have been calculated by way of example for a nucleic acid with a length of approx. 100 nucleotides and a G+C content of 50% in the absence of formamide. The experimental conditions for DNA hybridization are described in specialist textbooks of genetics such as, for example, in Sambrook et al., “Molecular Cloning”, Cold Spring Harbor Laboratory, 1989, and can be calculated using formulae with which the skilled worker is familiar, for example as a function of the length of the nucleic acids, the type of the hybrids or the G+C content. The skilled worker will find further information on hybridization in the following textbooks: Ausubel et al. (eds), 1985, “Current Protocols in Molecular Biology”, John Wiley & Sons, New York; Hames and Higgins (eds), 1985, “Nucleic Acids Hybridization: A Practical Approach”, IRL Press at Oxford University Press, Oxford; Brown (ed), 1991, Essential Molecular Biology: A Practical Approach, IRL Press at Oxford University Press, Oxford.

A functional equivalent is also understood as meaning, in particular, natural or artificial mutations of an originally isolated sequence encoding a dehydroquinate dehydratase/shikimate dehydrogenase, which continues to show the desired function. Mutations encompass substitutions, additions, deletions, exchanges or insertions of one or more nucleotide residues. Thus, the present invention also encompasses those nucleotide sequences which are obtained by modification of this nucleotide sequence. The purpose of such a modification may be, for example, the further delimitation of the coding sequence which it contains, or else, for example, the insertion of further cleavage sites for restriction enzymes.

Functional equivalents are also those variants whose function is reduced or increased compared with the original gene or gene fragment.

The term functional equivalent also covers the possibility that the nucleotide sequence according to the invention can be generated synthetically or obtained naturally or can comprise a mixture of synthetic and natural DNA components. In general, synthetic nucleotide sequences containing codons which are preferred by the host organism in question are generated. These preferred codons can be determined from codons with the highest protein frequency and which are expressed in most of the species of interest.

Functional analogs, or functional equivalents, of the nucleic acid sequences furthermore also encompass nucleic acid sequences which, based on the total length of the DNA sequence, have advantageously 40 to 100%, preferably 60 to 100%, especially preferably 70 to 100%, very especially preferably 80-100%, or 85-100%, or 90-100%, or 95-100%, or 96-100%, or 97-100%, or 98-100%, or 99-100% sequence homology with the DNA sequence SEQ ID No. 1 or SEQ ID No. 3.

The method according to the invention can be carried out in individual, separate steps; however, carrying out a high-throughput screening is preferred.

The abovementioned method allows the identification of herbicidally active substances which reduce or block the transcription, expression, translation or activity of a polypeptide with dehydroquinate dehydratase/shikimate dehydrogenase activity. These substances are potential herbicides whose effect can be improved further by traditional chemical synthesis.

Assay systems which are suitable for this purpose are both in-vitro and in-vivo assay systems.

Proteins which can be used for generating a test system for identifying substances which inhibit plant dehydroquinate dehydratase/shikimate dehydrogenase are proteins with dehydroquinate dehydratase/shikimate dehydrogenase activity which preferably

a) comprise the amino acid sequence shown in SEQ-ID No. 2 or SEQ-ID No. 4; or

b) comprise an amino acid part-sequence of at least 100 amino acids of SEQ ID No. 2 or SEQ ID No. 4 as claimed in claim 5.

The enzyme quantities required for the in-vitro assay systems are preferably provided via the functional expression of plant dehydroquinate dehydratase/shikimate dehydrogenase, in particular from tobacco dehydroquinate dehydratase/shikimate dehydrogenase, in suitable expression systems. However, the enzyme which has been isolated from plants, preferably from tobacco, may also be used in place of the recombinantly produced enzyme.

However, transgenic organisms are also preferably used for in-vivo assay systems.

Thus, a nucleic acid sequence such as the DNA sequence SEQ ID No. 1 or SEQ ID No. 3 comprising the coding region of a plant dehydroquinate dehydratase/shikimate dehydrogenase, or a nucleic acid sequence which hybridizes with the DNA sequence SEQ ID No. 1 or SEQ ID No. 3 or parts or derivatives derived from these sequences by insertion, deletion or substitution and which encodes a protein which has the biological activity of a plant dehydroquinate dehydratase/shikimate dehydrogenase can use for the introduction into prokaryotic or eukaryotic cells in in-vivo and in-vitro assay systems, this sequence optionally being linked to signal elements which ensure the transcription and translation in the cells and causing the expression of a translatable mRNA which brings about the synthesis of a plant dehydroquinate dehydratase/shikimate dehydrogenase.

The invention furthermore relates to expression cassettes whose sequence encode a tobacco dehydroquinate dehydratase/shikimate dehydrogenase or a functional equivalent thereof for generating an assay system for finding herbicidally active compounds. The nucleic acid sequence may be

a) a nucleic acid sequence with the sequence shown in SEQ ID No. 1 or SEQ ID No. 3; or

b) a nucleic acid sequence which, owing to the degeneracy of the genetic code, can be deduced from the amino acid sequences shown in SEQ ID No. 2 or SEQ ID No. 4 by backtranslation; or

c) functional analogs of the nucleic acid sequences shown in SEQ ID No. 1 or SEQ ID No. 3 which encode a polypeptide with the amino acid sequences shown in SEQ ID No. 2 or SEQ ID No. 4; or

d) functional analogs of the nucleic acid sequence shown in SEQ ID No. 1 or SEQ ID No. 3 which encode functional analogs of the amino acid sequences shown in SEQ ID No. 2 or SEQ ID No. 4; or

e) parts of the nucleic acid sequences a), b), c) or d); or

f) at least 300 nucleotide units of the nucleic acid sequences a), b), c) or d); and

g) optionally further regulatory elements.

Others which are suitable are artificial DNA sequences as long as they confer, as described for example above, the desired property of expressing the dehydroquinate dehydratase/shikimate dehydrogenase gene. Such artificial DNA sequences can be determined for example by backtranslating proteins constructed by means of molecular modeling which have dehydroquinate dehydratase/shikimate dehydrogenase activity, or else by in-vitro selection. Especially suitable are coding DNA sequences which were obtained by backtranslating a polypeptide sequence in accordance with the codon usage specific for the host organism. The specific codon usage can be determined readily by a skilled worker familiar with genetic methods by subjecting other, known genes of the organism to be transformed to computer evaluations. This methodology can also be used in expressing the target protein in bacteria, fungi, plants, insect cells and mammalian cells.

When preparing an expression cassette, various DNA fragments can be manipulated in order to obtain a nucleotide sequence which expediently reads in the correct direction and which is equipped with a correct reading frame. Adapters or linkers can be added to the fragments to connect the DNA fragments to one another. This methodology can be used as well in the expression of the target protein bacteria, fungi, plants, insect cells and mammalian cells.

As already mentioned, the abovementioned optionally additionally also contain what are known as regulatory nucleic acid sequences, also referred to as genetic functional elements, regulatory sequences, control sequences or control elements. Genetic functional elements are understood as meaning all those sequences which govern the expression of the coding sequence in the host cell. In accordance with a preferred embodiment, an expression cassette according to the invention comprises a promoter upstream, i.e. at the 5′ end of the coding sequence, and a terminator and optionally a polyadenylation signal downstream, i.e. at the 3′ end, and, if appropriate, further regulatory elements which are linked operably with the interposed sequence encoding the polypeptide with dehydroquinate dehydratase/shikimate dehydrogenase activity. Operable linkage is understood as meaning the sequential arrangement of promoter, coding sequence, terminator and, if appropriate, further regulating elements in such a way that each of the regulating elements can fulfil its intended function when the coding sequence is expressed.

Such an expression cassette is generated by fusing a suitable promoter, or a genetic control sequence, with a suitable dehydroquinate dehydratase/shikimate dehydrogenase DNA sequence and a polyadenylation signal, using customary recombination and cloning techniques as are described, for example, in T. Maniatis, E. F. Fritsch and J. Sambrook, Molecular Cloning: A Laboratory Manual, Cold Spring Harbor Laboratory, Cold Spring Harbor, N.Y. (1989) and in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., Current Protocols in Molecular Biology, Greene Publishing Assoc. and Wiley-Interscience (1987).

Genetic control sequences also encompass further promoters, promoter elements or minimal promoters capable of modifying the expression-governing properties. Thus, tissue-specific expression can additionally depend on certain stress factors, owing to genetic control sequences. Such elements have been described, for example, for water stress, abscisic acid (Lam E and Chua N H, J Biol Chem 1991; 266(26): 17131-17135) and heat stress (Schoffl F et al., Molecular & General Genetics 217(2-3):246-53, 1989).

Examples of advantageous control sequences for the expression cassettes or vectors according to the invention are, for example, in promoters such as cos, tac, trp, tet, lpp, lac, lacIq, T7, T5, T3, gal, trc, ara, SP6, I-PR or in the 1-PL promoter, all of which can be used for expressing dehydroquinate dehydratase/shikimate dehydrogenase in Gram-negative bacterial strains.

Further advantageous control sequences are present, for example, in the promoters amy and SPO₂, both of which can be used for expressing dehydroquinate dehydratase/shikimate dehydrogenase in Gram-positive bacterial strains, and in the yeast or fungal promoters ADC1, MFa, AC, P-60, CYC1, GAPDH, TEF, rp28, ADH, AOX1 and GAP, all of which can be used for expressing dehydroquinate dehydratase/shikimate dehydrogenase in yeast strains.

A promoter which is suitable for expression in plants is, in principle, any promoter capable of controlling the expression of foreign genes in plants. A plant promoter or a promoter derived from a plant virus is preferably used. Particularly preferred is the cauliflower mosaic virus CaMV ³⁵S promoter, see Franck et al., Cell 21, 285-294(1980). This promoter comprises a variety of recognition sequences for transcriptional effectors, which, in their totality, lead to permanent and constitutive expression of the gene which has been introduced, Benfey et al., EMBO J., 8, 2195-2202 (1989).

The expression cassette to be used for plants may also comprise a chemically inducible promoter, by means of which the expression of the exogenous dehydroquinate dehydratase/shikimate dehydrogenase gene can be controlled in the plant at a particular point in time. Such promoters, such as, for example, the PRP1 promoter (Ward et al., Plant. Mol. Biol. 22, 361-366(1993)), a salicylic-acid-inducible promoter (WO 95/19443), a benzenesulfonamide-inducible promoter (EP 0 388 186), a tetracyclin-inducible promoter (Gatz et al., Plant J. 2, 397-404(1992)), an abscisic-acid-inducible promoter (EP 0 335 528) or an ethanol- or cyclohexanone-inducible promoter (WO 93/21334) have been described in the literature and may be used, inter alia.

Other advantageous plant promoters are the promoter of the Glycine max phosphoribosylpyrophosphate amidotransferase (see also Genbank Accession Number U87999) or a node-specific promoter, such as in EP 249676.

Especially preferred promoters are furthermore those which ensure expression in tissues or plant parts in which the biosynthesis of amino acids or their precursors takes place. Promoters which ensure leaf-specific expression must be mentioned in particular. Promoters which must be mentioned are the potato cytosolic FBPase promoter or the potato ST-LSI promoter (Stockhaus et al., EMBO J. 8 (1989), 2445-245).

A foreign protein can be expressed stably in the seeds of transgenic tobacco plants to an extent of 0.67% of the total soluble seed protein with the aid of a seed-specific promoter (Fiedler and Conrad, Bio/Technology 10, 1090-1094 (1995)). The expression cassette according to the invention can therefore contain, for example, a seed-specific promoter (preferably the phaseolin promotor (U.S. Pat. No. 5,504,200), the USP promoter (USP=unknown seed protein, Baeumlein et al., Mol Gen Genet, 1991, 225 (3):459-67), the napin or the LEB4 promoter, or the promoter of the Arabidopsis oleosin gene (WO98/45461)), the LEB4 signal peptide (Baeumlein et al., 1992, Plant Journal, 2 (2):233-9), the gene to be expressed and an ER retention signal.

Other advantageous seed-specific promoters which can be used for monocotyledonous and dicotyledenous plants are promoters such as the oilseed rape napin gene promoter (U.S. Pat. No. 5,608,152), the Arabidopsis oleosin promoter (WO 98/45461), the Phaseolus vulgaris phaseolin promoter (U.S. Pat. No. 5,504,200), the Brassica Bce4 promoter (WO91/13980) or the B4 promoter from legumes (LeB4, Baeumlein et al., Plant J., 2, 2, 1992: 233-239) or promoters which are suitable for monocotyledonous plants, such as the promoters the promoters of the barley lpt2 or lpt1 gene (WO95/15389 and WO95/23230) or the promoters of the barley hordein gene, the rice glutelin gene, the rice oryzin gene, the rice prolamin gene, the wheat gliadin gene, the wheat glutelin gene, the maize zein gene, the oat glutelin gene, the sorghum kasirin gene or the rye secalin gene, which are described in WO99/16890.

The biosynthesis site of amino acids is generally the leaf tissue, so that leaf-specific expression of the dehydroquinate dehydratase/shikimate dehydrogenase gene makes sense. However, it is obvious that the amino acid biosynthesis need not be restricted to the leaf tissue, but can also take place in all remaining parts of the plant in a tissue-specific manner, for example in fatty seeds.

When generating expression cassettes which are suitable for the generation of transgenic plants, further regulatory sequences which ensure targeting into the apoplasts, into plastids, into the vaucole, into the mytochondrion, into the endoplasmic reticulum (ER) or which, owing to the absence of suitable operative sequences, ensure that the product remains in the compartment of its origin, namely the zytosol, especially preferably those which ensure targeting into plastids, are especially preferred; see Kermode, Crit. Rev. Plant Sci. 15(4), 285-423(1996).

It is also possible to construct expression cassettes, for expression in plants, whose DNA sequence encodes a dehydroquinate dehydratase/shikimate dehydrogenase fusion protein, part of the fusion protein being a transit peptide which governs the translocation of the polypeptide. Preferred are chloroplast-specific transit peptides which, following translocation of the dehydroquinate dehydratase/shikimate dehydrogenase gene into the chloroplasts, are enzymatically cleaved from the dehydroquinate dehydratase/shikimate dehydrogenase moiety. Especially preferred is the transit peptide which is derived from the plastid dehydroquinate dehydratase/shikimate dehydrogenase or a functional equivalent of this transit peptide (for example the transit peptide of the small Rubisco subunit or of ferrodoxin NADP oxidoreductase).

Attachment of the specific ER retention signal SEKDEL may also be of importance for the success according to the invention, see Schouten, A. et al., Plant Mol. Biol. 30, 781-792(1996); it triples to quadruples the average expression level. Other retention signals which occur naturally in plant and animal proteins which are localized in the ER may also be employed for constructing the cassette.

For example, a plant expression cassette according to the invention may comprise a constitutive promoter (preferably the CaMV 35S promoter), the LeB4 signal peptide, the gene to be expressed and the ER retention signal. The amino acid sequence KDEL (lysin, aspartic acid, glutamic acid, leucin) is preferably used as ER retention signal. Moreover, the plant expression cassette can be incorporated into, for example, the plant transformation vector pBinAR.

Thus, constitutive expression of the exogenous dehydroquinate dehydratase/shikimate dehydrogenase gene may generally be advantageous. However, inducible expression may also be desirable.

Moreover, further promoters may be linked operably to the nucleic acid sequence to be expressed, which promoters make possible expression in other plant tissues or in other organisms such as, for example, in E. coli bacteria. Suitable plant promoters are, in principle, all of the above-described promoters.

In a plant expression cassette which may optionally comprise polyadenylation signals, preferred polyadenylation signals are those which correspond essentially to T-DNA polyadenylation signals from Agrobacterium tumefaciens, in particular of gene 3 of the T-DNA (octopine synthase) of the Ti plasmid pTiACH5 (Gielen et al., EMBO J., 3, 835(1984)) or functional equivalents.

In an expression cassette according to the invention, the promoter and terminator regions can optionally be provided, in the direction of transcription, with a linker or polylinker containing one or more restriction sites for insertion of this sequence. As a rule, the linker has 1 to 10, in most cases from 1 to 8, preferably 2 to 6, restriction sites. In general, the linker within the regulatory regions has a size of less than 100 bp, frequently less than 60 bp, and at least 5 bp. The promoter according to the invention can be native or homologous, or else foreign or heterologous, to the host plant. The expression cassette according to the invention comprises, in the 5′-3′ direction of transcription, the promoter according to the invention, any sequence and a region for transcriptional termination. Various termination regions can be exchanged for each other as desired.

Manipulations which provide suitable restriction cleavage sites or which eliminate the excess DNA or restriction cleavage sites may also be employed. In-vitro mutagenesis, primer repair, restriction or ligation may be used in cases where insertions, deletions or substitutions such as, for example, transitions and transversions, are suitable. Complementary ends of the fragments may be provided for ligation in the case of suitable manipulations such as, for example, restriction, chewing-back or filling up overhangs for blunt ends.

To transform a host plant with a DNA encoding a dehydroquinate dehydratase/shikimate dehydrogenase, an expression cassette is incorporated, as an insertion, into a vector whose vector DNA contains additional functional regulatory signals, for example sequences for replication or integration.

In addition to plasmids, vectors are also to be understood as including all of the other vectors with which the skilled worker is familiar, such as, for example, phages, viruses such as SV40, CMB, baculovirus, adenovirus, transponsons, IS elements, phasmids, phagemids, cosmids, or linear or circular DNA. These vectors are capable of autonomous replication in the host organism or of chromosomal replication; chromosomal replication is preferred.

In a further embodiment of the vector, the nucleic acid construct can advantageously also be introduced into the organisms in the form of a linear DNA and integrated into the genome of the host organism via heterologous or homologous recombination. This linear DNA may consist of a linearized plasmid or just of the nucleic acid construct as vector, or the nucleic acid sequences used.

In a further advantageous embodiment, the nucleic acid sequences used in the method according to the invention may also be introduced into an organism by themselves.

If, in addition to the nucleic acid sequences, further genes are to be introduced into the organism, it is possible to introduce all of them together in a single vector into the organism, or to introduce each individual gene into the organism in one vector each, it being possible to introduce the various vectors simultaneously or in succession.

The vector advantageously comprises at least one copy of the nucleic acid sequences used and/or of the nucleic acid construct according to the invention.

In addition to the abovementioned promoters, the expression cassettes according to the invention and the vectors derived from them may also comprise further functional elements, as already suggested above. Examples which may be mentioned, but not by limitation, are:

1. reporter genes encoding readily quantifiable proteins. An assessment of the transformation efficiency or of the site or timing of expression can be performed by means of these genes via growth, fluorescence, chemoluminescence, bioluminescence or resistance assay or via a photometric measurement (intrinsic color) or enzyme activity. Very especially preferred are reporter proteins in this context (Schenborn E, Groskreutz D. Mol Biotechnol. 1999; 13(1):29-44), such as the “green fluorescence protein” (GFP) (Gerdes H H and Kaether C, FEBS Lett. 1996; 389(1):44-47; Chui W L et al., Curr Biol 1996, 6:325-330; Leffel S M et al., Biotechniques. 23(5):912-8, 1997), chloramphenicol acetyltransferase, a luciferase (Giacomin, Plant Sci 1996, 116:59-72; Scikantha, J Bact 1996, 178:121; Millar et al., Plant Mol Biol Rep 1992 10:324-414), and luciferase genes, the β-galactosidase gene or the β-glucuronidase gene (Jefferson et al., EMBO J. 1987, 6, 3901-3907), the the Ura3 gene, the Ilv2 gene, the 2-deoxyglucose-6-phosphate phosphatase gene, the b-lactamase gene, the neomycin phosphotransferase gene, the hygromycin phosphotransferase gene or the BASTA (=glufosinate resistance) gene;

2. replication origins;

3. selection markers which confer resistance to antibiotics. Examples which may be mentioned in this context are the npt gene, which confers resistance to the aminoglycoside antibiotics neomycin (G 418), kanamycin, and paromycin (Deshayes A et al., EMBO J. 4 (1985) 2731-2737), the hygro gene (Marsh J L et al., Gene. 1984; 32(3):481-485), the sul gene (Guerineau F et al., Plant Mol. Biol. 1990; 15(1):127-136) and the she-ble gene, which confers resistance to the bleomycin antibiotic zeocin. Further examples of selection marker genes are genes which confer resistance to 2-deoxyglucose-6-phosphate (WO 98/45456) or phosphinothricin and the like, or those which confer resistance to antimetabolites, for example the dhfr gene (Reiss, Plant Physiol. (Life Sci. Adv.) 13 (1994) 142-149). Other suitable genes are genes like trpB or hisD (Hartman SC and Mulligan RC, Proc Natl Acad Sci USA. 85 (1988) 8047-8051). Another suitable gene is the mannose phosphate isomerase gene (WO 94/20627), the ODC (ornithin decarboxylase) gene (McConlogue, 1987 in: Current Communications in Molecular Biology, Cold Spring Harbor Laboratory, ed.) or the Aspergillus terreus deaminase (Tamura K et al., Biosci Biotechnol Biochem. 59 (1995) 2336-2338).

4. What are known as affinity tags, which encode a peptide or polypeptide whose nucleic acid sequence can be fused with the sequence encoding the target protein either directly or by means of a linker, using customary cloning techniques. The affinity tag is used for isolating the recombinant target protein by means of affinity chromatography, but, under certain circumstances, it may also be used for detecting the expressed fusion protein. The abovementioned linker may optionally comprise a protease cleavage site (for example for thrombin or factor Xa), by means of which the affinity tag can be cleaved from the target protein if so desired. Examples of current affinity tags are the “His tag” for example from Quiagen, Hilden, the “Strep tag”, the “Myc tag”, the tag from New England Biolab, which consists of a chitin-binding domain and an intein, and what is known as the CBD tag from Novagen.

The use of expression systems and vectors which are available to the public or commercially available is furthermore also possible for expressing the dehydroquinate dehydratase/shikimate dehydrogenase. The following enumeration is by way of example, but not by limitation.

Examples of vectors of vectors for the expression in E. coli are PGEX [Pharmacia Biotech Inc; Smith, D. B. and Johnson, K. S. (1988) Gene 67:31-40], pMAL (New England Biolabs, Beverly, Mass.) and pRIT5 (Pharmacia, Piscataway, N.J.), which comprises glutathione S-transferase (GST), maltose binding protein, or protein A, the pTrc vectors (Amann et al., (1988) Gene 69:301-315), the “pQE” vectors from Qiagen (Hilden), “pKK233-2” from CLONTECH, Palo Alto, Calif. and the “pET” and the “pBAD” vector series from Stratagene, La Jolla, and the M13 mp series and pACYC184.

Examples of vectors for use in yeast are pYepSecl (Baldari, et al., (1987) Embo J. 6:229-234), pMFa (Kurjan and Herskowitz, (1982) Cell 30:933-943), pJRY88 (Schultz et al., (1987) Gene 54:113-123), and pYES derivatives, pGAPZ derivatives, pPICZ derivatives, and the vectors of the “Pichia Expression Kit” (all from Invitrogen Corporation, San Diego, Calif.).

Examples of vectors for use in filamentous fungi are described in: van den Hondel, C. A. M. J. J. & Punt, P. J. (1991) “Gene transfer systems and vector development for filamentous fungi, in: Applied Molecular Genetics of Fungi”, J. F. Peberdy, et al., eds., p. 1-28, Cambridge University Press: Cambridge.

Examples of insect cell expression vectors, for example for the expression in Sf9 cells, are the vectors of the pAc series (Smith et al. (1983) Mol. Cell Biol. 3:2156-2165) and of the pVL series (Lucklow and Summers (1989) Virology 170:31-39).

Examples of plant expression vectors for the expression in plant cells or algal cells are found in Becker, D., et al. (1992) “New plant binary vectors with selectable markers located proximal to the left border”, Plant Mol. Biol. 20: 1195-1197 or in Bevan, M. W. (1984) “Binary Agrobacterium vectors for plant transformation”, Nucl. Acid. Res. 12: 8711-8721. Further suitable vectors are described, inter alia, in “Methods in Plant Molecular Biology and Biotechnology” (CRC Press, chapter 6/7, 71-119).

Examples of expression vectors to be used in mammalian cells are pCDM8 and pMT2PC, which are mentioned in: Seed, B. (1987) Nature 329:840 or Kaufman et al. (1987) EMBO J. 6:187-195). Promoters which are to be used by preference are of viral origin, such as, for example, promoters of the polyoma virus, adenovirus 2, cytomegalovirus or Simian Virus 40. Further prokaryotic or eukaryotic expression systems are mentioned in chapters 16 and 17 in Sambrook et al., Molecular Cloning: A Laboratory Manual. 2nd, ed., Cold Spring Harbor Laboratory, Cold Spring Harbor Laboratory Press, Cold Spring Harbor, N.Y., 1989. Further advantageous vectors are described in Hellens et al. (Trends in plant science, 5, 2000).

Moreover, the expression cassette and the vectors derived therefrom can be employed for transforming bacteria, cyanobacteria, yeasts, filamentous fungi and algae with the purpose of increasing the content in ubiquinone, folate, flavonoids, coumarins, lignins, alkaloids, cyanogenic glycosides, plastoquinones, tocopherols and aromatic amino acids.

Preferred within the bacteria are bacteria of the genus Escherichia (Escherichia coli), Erwinia, Flavobacterium, Alcaligenes or cyano bacteria, for example of the genus Synechocystis or Anabena. Bacteria of the genus Escherichia coli are especially preferred in this context for economic reasons and because of the multiplicity of possible genetic manipulations. Preferred yeasts are Candida, Saccharomyces, Hansenula or Pichia. Preferred fungi are Aspergillus, Trichoderma, Ashbya, Mortierella, Saprolegnia, Pythium, Neurospora, Fusarium, Beauveria or further fungi described in Indian Chem Engr. Section B. Vol 37, No 1,2 (1995). Preferred eukaryotic cell lines are, for example, customary insect or mammalian cell lines with which the skilled worker is familiar. In principle, transgenic animals, for example C. elegans, are also suitable as host organisms.

Furthermore preferred are transgenic plants comprising a functional or nonfunctional nucleic acid construct according to the invention or a functional or nonfunctional vector according to the invention. Functional means, for the purposes of the invention, that the nucleic acids used in the methods are expressed alone or in the nucleic acid construct or in the vector and that a biologically active gene product is generated. Nonfunctional means, for the purposes of the invention, that the nucleic acids used in the method are not transcribed or not expressed alone or in the nucleic acid construct or in the vector and/or that a biologically inactive gene product is generated. In this sense, what are known as antisense RNAs are also nonfunctional nucleic acids or, in the case of insertion into the nucleic acid construct or the vector, a nonfunctional nucleic acid construct or nonfunctional vector. Both the nucleic acid construct according to the invention and the vector according to the invention can be used advantageously for the generation of transgenic organisms, preferably plants.

Also preferred is the use of commercially available systems for expressing the recombinant dehydroquinate dehydratase/shikimate dehydrogenase, such as, for example, the baculovirus expression systems “MaxBac 2.0 Kit” from Invitrogen, Carlsbad, or the “BacPAK Baculovirus Expression System” from CLONTECH, Palo Alto, Calif., expression systems for yeasts, such as the “Easy Select Pichia Expression Kit”, the “Pichia Expression Kit” (all from Invitrogen, Carlsbad) or the “Yeast Protein Expression and Purification System” from Stratagene, La Jolla.

The plant dehydroquinate dehydratase/shikimate dehydrogenase protein which is expressed with the aid of an expression cassette is particularly suitable for finding, in in-vitro assay systems, inhibitors which are specific for dehydroquinate dehydratase/shikimate dehydrogenase. To this end, for example, the cDNA sequence of dehydroquinate dehydratase/shikimate dehydrogenase or suitable fragments of the cDNA sequence of dehydroquinate dehydratase/shikimate dehydrogenase from tobacco can be cloned in one of the abovementioned expression vectors, such as, for example, the vector pQE, and overexpressed in one of the abovementioned organisms or expression systems, such as, for example, E. coli, since E. coli is particularly suitable for the expression of recombinant proteins, for the above-mentioned reasons.

In principle, the method according to the invention for the identification of herbicidally active inhibitors of a polypeptide with dehydroquinate dehydratase/shikimate dehydrogenase activity is based on influencing the transcription, expression, translation or the activity of the gene product of the amino acid sequence encoded by a nucleic acid sequence selected from the group:

a) a nucleic acid sequence with the sequence shown in SEQ ID No. 1 or SEQ ID No. 3; or

b) a nucleic acid sequence which, owing to the degeneracy of the genetic code, can be deduced from the amino acid sequences shown in SEQ ID No. 2 or SEQ ID No. 4 by backtranslation; or

c) functional analogs of the nucleic acid sequences shown in SEQ ID No. 1 or SEQ ID No. 3 which encode a polypeptide with the amino acid sequences shown in SEQ ID No. 2 or SEQ ID No. 4; or

d) functional analogs of the nucleic acid sequence shown in SEQ ID No. 1 or SEQ ID No. 3 which encode functional analogs of the amino acid sequences shown in SEQ ID No. 2 or SEQ ID No. 4; or

e) parts of the nucleic acid sequences a), b), c) or d); or

f) at least 300 nucleotide units of the nucleic acid sequences a), b), c) or d);

and selecting those substances which reduce or block the transcription, expression, translation or the activity of the gene product.

As has already been mentioned above, carrying out these assays in a high-throughput screening system is particularly advantageous.

To verify the herbicidal properties of a substance identified via the method according to the invention, the procedure of choice would be to assay the herbicidal properties by applying the substances to a plant and to compare said plant with a plant which has not been incubated with a substance identified via the method.

In a preferred embodiment, the method is carried out in an organism, the organism used being bacteria, yeasts, fungi or plants. In this context, it is possible to use an organism which is a conditional or natural mutant of the sequence SEQ ID No. 1 or SEQ ID No. 3. Especially preferred is a method in which the organism employed is a transgenic organism.

The term transgenic organism refers in the present context to an organism which has been transformed with an expression cassette according to the invention or with a vector according to the invention. The transfer of foreign genes into the genome of an organism is referred to as transformation in this context.

A series of standard procedures for the transformation of a range of organisms are known to the skilled worker (Sambrook et al., Cold Spring Harbor Laboratory Press (1989) and Ausubel, F. M. et al., Current Protocols in Molecular Biology, Greene Publishing Assoc. and Wiley-Interscience (1994) ISBN 0-87969-309-6).

Some of the transformation procedures used for plants will now be illustrated briefly in the following text:

To transform plants, the above-described methods for the transformation and regeneration of plants from plant tissues or plant cells can be exploited for transient or stable transformation. Suitable methods are protoplast transformation by polyethylene-glycol-induced DNA uptake, the biolistic approach with the gene gun, electroporation, incubation of dry embryos in DNA-containing solution, microinjection and agrobacteria-mediated gene transfer. The abovementioned methods are described in, for example, B. Jenes et al., Techniques for Gene Transfer, in: Transgenic Plants, Vol. 1, Engineering and Utilization, edited by S. D. Kung and R. Wu, Academic Press (1993), 128-143 and in Potrykus Annu. Rev. Plant Physiol. Plant Molec. Biol. 42, 205-225(1991). A further method for the generation of transgenic plants, with which method the skilled worker is familiar, is what is known as plastid transformation. A review regarding customary suitable techniques is described in Aart van Bel et al., Curr. Op. Bitechmol (2001)12 144-149.

Preferably, an expression cassette according to the invention which encodes a dehydroquinate dehydratase/shikimate dehydrogenase gene is cloned into a vector, for example pBINAR, which vector is suitable for the transformation of Agrobacterium tumefaciens, for example pBinl9 (Bevan et al., Nucl. Acids Res. 12, 8711(1984)). Agrobacteria transformed with such a vector can then be used in the known manner for transforming plants, in particular crop plants, such as, for example, tobacco plants, for example by bathing scarified leaves or leaf sections in an agrobacterial solution and subsequently growing them in suitable media. The transformation of plants by agrobacteria is known, inter alia, from F. F. White, Vectors for Gene Transfer in Higher Plants; in Transgenic Plants, Vol. 1, Engineering and Utilization, edited by S. D. Kung and R. Wu, Academic Press, 1993, pp. 15-38. Transgenic plants which comprise a gene for the expression of a dehydroquinate dehydratase/shikimate dehydrogenase gene integrated into the expression cassette can be regenerated from the transformed cells of the scarified leaves or leaf sections in the known manner.

Agrobacteria transformed with an expression cassette can equally be used in a known manner for transforming plants, in particular crop plants such as cereals, maize, soybean, rice, cotton, sugarbeet, canola, sunflower, flax, hemp, potato, tobacco, tomato, oilseed rape, alfalfa, lettuce and the various tree, nut and grapevine species, and also legumes, for example by bathing scarified leaves or leaf sections in an agrobacterial solution and subsequently culturing them in suitable media.

As already mentioned briefly above, the invention furthermore relates to in-vitro methods for identifying herbicidally active substances which inhibit the activity of the plant dehydroquinate dehydratase/shikimate dehydrogenase.

In a preferred embodiment, the method according to the invention consists of the following steps:

a) a polypeptide with dehydroquinate dehydratase/shikimate dehydrogenase activity is either expressed in enzymatically active form in one of the above-described embodiments of a transgenic organism, or an organism comprising the protein according to the invention is cultured;

b) the protein obtained in step a) is incubated with redox equivalents and with a chemical compound either in the growing or quiescent organism as a whole, in the cell digest of the transgenic organism, in partially purified form or in homogeneously purified form; all of the redox equivalents known to the skilled worker may be used for this purpose. Examples which may be mentioned, but not by limitation, are: NADPH/NADP+, NADH/NAD+ and FAD/FADH.

c) a chemical comopound is selected by step b) which inhibits a polypeptide with dehydroquinate dehydratase/shikimate dehydrogenase activity in comparison with a sample which has not been incubated with the chemical compound.

This method is particularly suitable for a high-throughput screening procedure.

In this method, the plant dehydroquinate dehydratase/shikimate dehydrogenase can be employed for example in an enzyme assay in which the activity of the dehydroquinate dehydratase/shikimate dehydrogenase is determined in the presence and absence of the active ingredient to be assayed. A qualitative and quantitative finding regarding the inhibitory behavior of the active ingredient to be assayed can be obtained by comparing the two activity determinations.

A large number of chemical compounds can be assayed rapidly and simply for herbicidal properties with the aid of the assay system according to the invention. The method allows reproducible selection, from a large number of substances, specifically of those which are very potent, in order to subsequently subject these substances to further in-depth tests with which the skilled worker is familiar.

In a further embodiment of the invention, inhibitors of the enzyme dehydroquinate dehydratase/shikimate dehydrogenase can be detected with the aid of techniques which indicate the interaction between protein and ligand. In this context, three preferred embodiments which are also suitable for high-throughput methods in connection with the present invention must be mentioned in particular:

a) the average diffusion rate of a fluorescent molecule as a function of mass can be determined in a small sample volume via fluorescence correlation spectroscopy (FCS) (Proc. Natl. Acad. Sci. USA (1994) 11753-11575). FSC can be employed for determining protein/ligand interactions by measuring the changes in the mass, or the changed diffusion rate, which this entails, of a chemical compound when binding to dehydroquinate dehydratase/shikimate dehydrogenase. The chemical compounds which are identified in this manner and which bind to dehydroquinate dehydratase/shikimate dehydrogenase may be suitable as inhibitors.

b) Surface-enhanced laser desorption/ionization (SELDI) in combination with a time-of-flight mass spectrometer (MALDI-TOF) makes possible the rapid analysis of molecules on a support and can be used for analysing protein-ligand interactions (Worral et al., (1998) Anal. Biochem. 70:750-756). In a preferred embodiment, dehydroquinate dehydratase/shikimate dehydrogenase is immobilized on a suitable support, which is then incubated with the chemical compound to be assayed. After one or more suitable wash steps, the molecules of the chemical compound which are additionally bound to the protein can be detected by means of the above-stated methodology, and thus possible inhibitors are selected. The chemical compounds which are identified in this manner and which bind to dehydroquinate dehydratase/shikimate dehydrogenase may be suitable as inhibitors.

c) Biacore is based on the change in the refractive index on a surface when a chemical compound binds to a protein immobilized on said surface. Since the change in the refractive index for a defined change in the mass concentration at the surface is virtually identical for all proteins and polypeptides, this method can be applied, in principle, to any protein (Lindberg et al. Sensor Actuators 4 (1983) 299-304; Malmquist Nature 361 (1993) 186-187). The chemical compound is injected into a cuvette with a volume of 2-5 ml at whose walls the protein has been immobilized. The binding of the chemical compound in question to the protein, and thus the identification of possible inhibitors, can be determined via surface plasmon resonance (SPR) by the absorption of the laser light reflected by the surface. The chemical compounds which are identified in this manner and which bind to dehydroquinate dehydratase/shikimate dehydrogenase may be suitable as inhibitors.

d) Furthermore, there exists the possibility of detecting further candidates for herbicidal active ingredients by molecular modeling via elucidation of the three-dimensional structure of dehydroquinate dehydratase/shikimate dehydrogenase by x-ray structure analysis. The preparation of protein crystals required for x-ray structure analysis, and the relevant measurements and subsequent evaluations of these measurements, and the methodology of molecular modeling are known to the skilled worker. In principle, an optimization of the compounds identified by the abovementioned methods is also possible via molecular modeling.

The invention furthermore relates to in-vivo methods of identifying herbicidally active substances which inhibit the dehydroquinate dehydratase/shikimate dehydrogenase activity in plants, consisting of

a) the generation of a transgenic organism comprising an expression cassette or vector according to the invention, which comprises an additional nucleic acid sequence encoding an enzyme with dehydroquinate dehydratase/shikimate dehydrogenase activity and which is capable of overexpressing an enzymatically active dehydroquinate dehydratase/shikimate dehydrogenase;

b) applying a substance to the transgenic organism;

c) determining the growth or the viability of the transgenic and the nontransgenic organism after application of the chemical substance; and

d) the comparison of the growth or the viability of the transgenic and the nontransgenic organism after application of the chemical substance;

The following organisms or cell types can be used for generating a transgenic organism: bacteria, yeasts, fungi, algae, plant cells, insect cells or mammalian cells.

Suppression of growth or viability of the nontransformed organism without the growth or the viability of the transgenic organism being affected confirms that the substance of b) inhibits the dehydroquinate dehydratase/shikimate dehydrogenase enzyme activity in plants and thus demonstrates herbicidal activity.

Chemical compounds which reduce the biological activity, the growth or the vitality of the organisms are understood as meaning compounds which inhibit the biological activity, the growth or the vitality of the organisms by at least 10%, advantageously by at least 30%, preferably by at least 50%, especially preferably by at least 70%, very especially preferably by at least 90%.

In a preferred embodiment of the abovementioned method, the transgenic organisms employed are transgenic plants, plant cells, plant tissues or plant parts.

The invention furthermore relates to herbicidally active compounds which can be identified with the above-described assay systems.

The invention furthermore relates to a method which consists in applying, to a plant, the substances identified via the abovementioned methods in order to assay their herbicidal activity and selecting those substances which demonstrate herbicidal activity.

The substances which have been identified can be chemically synthesized substances or substances produced by microorganisms and can be found, for example, in cell extracts of, for example, plants, animals or microorganisms. Furthermore, the substances mentioned may be known in the prior art, but as yet unknown as herbicides. The reaction mixture can be a cell-free extract or comprise a cell or cell culture. Suitable methods are known to the skilled worker and are described generally for example in Alberts, Molecular Biology the cell, 3rd Edition (1994), for example chapter 17. The substances mentioned can be added to, for example, the reaction mixture or the culture medium or injected into the cells or sprayed onto a plant.

When a sample which contains an active substance which has been detected by the method according to the invention, then one possibility is to isolate the substance directly from the original sample. As an alternative, the sample can be divided into various groups, for example when it consists of a multiplicity of different components, in order to reduce the number of different substances per sample and then to repeat the method according to the invention with such a “subsample” of the original sample. Depending on the complexity of the sample, the above-described steps can be repeated many times, preferably until the sample identified in accordance with the method according to the invention only contains a small number of substances, or just one substance. Preferably, the substance identified in accordance with the method according to the invention or derivatives thereof are formulated further so that it is suitable for use in plant breeding, plant cell culture or tissue culture.

The substances which have been assayed and identified in accordance with the method according to the invention may be expression libraries, for example cDNA expression libraries, peptides, proteins, nucleic acids, antibodies, small organic substances, hormones, PNAs or the like (Milner, Nature Medicin 1 (1995), 879-880; Hupp, Cell. 83 (1995), 237-245; Gibbs, Cell. 79 (1994), 193-198 and references cited therein). These substances can also be functional derivatives or analogs of the known inhibitors or activators. Methods for preparing chemical derivatives or analogs are known to the skilled worker. The abovementioned derivatives and analogs can be assayed in accordance with prior-art methods. Moreover, computer-aided design or peptidomimetics may be used for preparing suitable derivatives and analogs. The cell or the tissue which can be used for the method according to the invention is preferably a host cell according to the invention, plant cell according to the invention or a plant tissue, as described in the above-mentioned embodiments.

A further embodiment of the invention are substances which have been identified by the above-described methods according to the invention, the substances taking the form of an antibody against the protein encoded by the sequence SEQ ID No. 1 or SEQ ID No. 3 or a functional equivalent of the protein encoded by the sequence SEQ ID No. 1 or SEQ ID No. 3.

Herbicidally active dehydroquinate dehydratase/shikimate dehydrogenase inhibitors can be used as defoliants, desiccants, haulm killers and, in particular, as weed killers. Weeds, in the broadest sense, are understood as meaning all plants which grow at locations where they are undesired. Whether the active ingredients found with the aid of the assay system according to the invention act as nonselective or selective herbicides depends, inter alia, on the amount used.

Herbicidally active dehydroquinate dehydratase/shikimate dehydrogenase inhibitors can be used for example against the following weeds:

Dicotyledonous weeds of the genera:

Sinapis, Lepidium, Galium, Stellaria, Matricaria, Anthemis, Galinsoga, Chenopodium, Urtica, Senecio, Amaranthus, Portulaca, Xanthium, Convolvulus, Ipomoea, Polygonum, Sesbania, Ambrosia, Cirsium, Carduus, Sonchus, Solanum, Rorippa, Rotala, Lindernia, Lamium, Veronica, Abutilon, Emex, Datura, Viola, Galeopsis, Papaver, Centaurea, Trifolium, Ranunculus, Taraxacum.

Monocotyledonous weeds of the genera:

Echinochloa, Setaria, Panicum, Digitaria, Phleum, Poa, Festuca, Eleusine, Brachiaria, Lolium, Bromus, Avena, Cyperus, Sorghum, Agropyron, Cynodon, Monochoria, Fimbristyslis, Sagittaria, Eleocharis, Scirpus, Paspalum, Ischaemum, Sphenoclea, Dactyloctenium, Agrostis, Alopecurus, Apera.

Depending on the application method in question, the substances identified in the method according to the invention, or compositions comprising them, can advantageously also be employed in a further number of crop plants for eliminating undesired plants. Suitable crops are, for example, the following:

Allium cepa, Ananas comosus, Arachis hypogaea, Asparagus officinalis, Beta vulgaris spec. altissima, Beta vulgaris spec. rapa, Brassica napus var. napus, Brassica napus var. napobrassica, Brassica rapa var. silvestris, Camellia sinensis, Carthamus tinctorius, Carya illinoinensis, Citrus limon, Citrus sinensis, Coffea arabica (Coffea canephora, Coffea liberica), Cucumis sativus, Cynodon dactylon, Daucus carota, Elaeis guineensis, Fragaria vesca, Glycine max, Gossypium hirsutum, (Gossypium arboreum, Gossypium herbaceum, Gossypium vitifolium), Helianthus annuus, Hevea brasiliensis, Hordeum vulgare, Humulus lupulus, Ipomoea batatas, Juglans regia, Lens culinaris, Linum usitatissimum, Lycopersicon lycopersicum, Malus spec., Manihot esculenta, Medicago sativa, Musa spec., Nicotiana tabacum (N. rustica), Olea europaea, Oryza sativa, Phaseolus lunatus, Phaseolus vulgaris, Picea abies, Pinus spec., Pisum sativum, Prunus avium, Prunus persica, Pyrus communis, Ribes sylvestre, Ricinus communis, Saccharum officinarum, Secale cereale, Solanum tuberosum, Sorghum bicolor (s. vulgare), Theobroma cacao, Trifolium pratense, Triticum aestivum, Triticum durum, Vicia faba, Vitis vinifera, Zea mays.

In addition, the substances found by the method according to the invention can also be used in crops which tolerate the action of herbicides owing to breeding, including recombinant methods.

The substances according to the invention, or the herbicidal compositions comprising them, can be formulated for example in the form of directly sprayable aqueous solutions, powders, suspensions, also highly concentrated aqueous, oily or other suspensions or dispersions, emulsions, oil dispersions, pastes, dusts, materials for spreading or granules by means of spraying, atomizing, dusting, spreading or pouring. The use forms depend on the intended use; in any case, they should ensure the finest possible distribution of the active ingredients according to the invention.

Suitable inert liquid and/or solid carriers are liquid additives such as mineral oil fractions of medium to high boiling point such as kerosene or diesel oil, furthermore coal tar oils and oils of vegetable or animal origin, aliphatic, cyclic and aromatic hydrocarbons, for example paraffin, tetrahydrophthalene, alkylated naphthalenes or their derivatives, alkylated benzenes or their derivatives, alcohols such as methanol, ethanol, propanol, butanol and cyclohexanol, ketones such as cyclohexanone, or strongly polar solvents, for example amines such as N-methylpyrrolidone or water.

Further advantageous use forms of the substances and/or compositions according to the invention are aqueous use forms such as emulsion concentrates, suspensions, pastes, wettable powders or water-dispersible granules, which can be prepared for example by adding water. To prepare emulsions, pastes or oil dispersions, the substances and/or compositions, what are known as substrates, can be homogenized in water by means of wetters, stickers, dispersants or emulsifiers, either as such or dissolved in an oil or solvent. It is also possible to prepare concentrates consisting of active substance, wetter, sticker, dispersant or emulsifier and, if appropriate, solvent or oil, and these concentrates are suitable for dilution with water.

Suitable surface-active substances are, for example, alkali metal salts, alkaline earth metal salts or ammonium salts of aromatic sulfonic acids, for example lignosulfonic acid, phenolsulfonic acid, naphthalenesulfonic acid and dibutylnaphthalenesulfonic acid, and of fatty acids, of alkyl- and alkylarylsulfonates, of alkyl sulfates, lauryl ether sulfates and fatty alcohol sulfates, and salts of sulfated hexa-, hepta- and octadecanols and of fatty alcohol glycol ethers, condensates of sulfonated naphthalene and its derivatives with formaldehyde, condensates of naphthalene or of the naphthalenesulfonic acids with phenol and formaldehyde, polyoxyethylene octylphenol ether, ethoxylated isooctyl-, octyl- or nonylphenol, alkylphenyl polyglycol ethers, tributylphenyl polyglycol ether, alkylaryl polyether alcohol, isotridecyl alcohol, fatty alcohol/ethylene oxide condensates, ethoxylated castor oil, polyoxyethylene alkyl ethers or polyoxypropylene alkyl ethers, lauryl alcohol polyglycol ether acetate, sorbitol esters, lignin-sulfite waste liquors or methylcellulose.

Powders, materials for spreading and dusts, as solid carriers, can be prepared advantageously by mixing or concomitantly grinding the active substances with a solid carrier.

Granules, for example coated granules, impregnated granules and homogeneous granules, can be prepared by binding the active ingredients to solid carriers. Examples of solid carriers are mineral earths such as silicas, silica gels, silicates, talc, kaolin, limestone, lime, chalk, bole, loess, clay, dolomite, diatomaceous earth, calcium sulfate, magnesium sulfate, magnesium oxide, ground synthetic materials, fertilizers such as ammonium sulfate, ammonium phosphate, ammonium nitrate, ureas and products of vegetable origin such as cereal meal, tree bark meal, wood meal and nutshell meal, cellulose powders or other solid carriers.

The concentrations of the substances and/or compositions according to the invention in the ready-to-use preparations can vary within wide ranges. In general, the formulations comprise 0.001 to 98% by weight, preferably 0.01 to 95% by weight, of at least one active ingredient. The active ingredients are employed in a purity of 90% to 100%, preferably 95% to 100% (according to MR spectrum).

The herbicidal compositions, or the substances, can be applied re- or post-emergence. If the active ingredients are less well tolerated by certain crop plants, application techniques may be used in which the compositions are sprayed, with the aid of the spraying apparatus, in such a way that they come into as little contact as possible, if any, with the leaves of the sensitive crop plants while the active ingredients reach the leaves of undesired plants which grow underneath, or the bare soil surface (post-directed, lay-by).

To widen the spectrum of action and to achieve synergistic effects, the substances and/or compositions according to the invention may be mixed with a large number of representatives of other groups of herbicidal or growth-regulatory active ingredients and applied concomitantly with them. Suitable components in mixtures are, for example, 1,2,4-thiadiazoles, 1,3,4-thiadiazoles, amides, aminophosphoric acid and its derivatives, aminotriazoles, anilides, (het)aryloxyalkanoic acids and their derivatives, benzoic acid and its derivatives, benzothiadiazinones, 2-aroyl-1,3-cyclohexanediones, hetaryl aryl ketones, benzylisoxazolidinones, meta-CF3-phenyl derivatives, carbamates, quinolincarboxylic acid and its derivatives, chloroacetanilides, cyclohexan-1,3-dione derivatives, diazines, dichloropropionic acid and its derivatives, dihydrobenzofurans, dihydrofuran-3-ones, dinitroanilines, dinitrophenols, diphenyl ethers, dipyridyls, halocarboxylic acids and their derivatives, ureas, 3-phenyluracils, imidazoles, imidazolinones, N-phenyl-3,4,5,6-tetrahydrophthalimides, oxadiazoles, oxiranes, phenols, aryloxy- or heteroaryloxyphenoxypropionic esters, phenylacetic acid and its derivatives, phenylpropionic acid and its derivatives, pyrazoles, phenylpyrazoles, pyridazines, pyridinecarboxylic acid and its derivatives, pyrimidyl ethers, sulfonamides, sulfonylureas, triazines, triazinones, triazolinones, triazolecarboxamides and uracils.

Moreover, it may be advantageous to apply the substances and/or compositions according to the invention, alone or in combination with other herbicides, jointly together with further crop protectants, for example with agents for controlling pests or phytopathogenic fungi or bacteria. Furthermore of interest is the miscibility with mineral salt solutions, which are employed for alleviating nutritional and trace element deficiencies. Nonphytotoxic oils and oil concentrates may also be added.

Depending on the intended aim, the season, the target plants and the growth stage, the application rates of active ingredient (=substances and/or compositions) amount to 0.001 to 3.0, preferably 0.01 to 1.0 kg/ha of active substance.

Another subject of the invention is the use of a substance identified by one of the methods according to the invention or compositions comprising these substances as herbicide or for regulating the growth of plants.

The invention furthermore relates to transgenic organisms, preferably plants, transformed with an expression cassette comprising the DNA sequence SEQ ID No. 1 or SEQ ID No. 3 or its functional equivalents, which plants, owing to the additional expression of the DNA sequence SEQ ID No. 1 or SEQ ID No. 3 or of a functional equivalent of one of these sequences, have been made tolerant to dehydroquinate dehydratase/shikimate dehydrogenase inhibitors, and to transgenic cells, tissues, parts and propagation material of such transgenic organisms, preferably plants. Especially preferred in this context are transgenic crop plants such as, for example, barley, wheat, rye, maize, soybean, rice, cotton, sugar beet, canola, sunflower, flax, hemp, potato, tobacco, tomato, oilseed rape, alfalfa, lettuce and the various tree, nut and grapevine species, and legumes.

The invention thus furthermore relates to the use of an expression cassette comprising DNA sequences SEQ ID No. 1, SEQ ID No. 3 or DNA sequences hybridizing with these for the transformation of plants, plant cells, plant tissues or plant parts. The preferred aim of the use is the generation of plants with herbicide-resistant forms of dehydroquinate dehydratase/shikimate dehydrogenase.

In a modified form, or in a form which leads to overexpression, the gene encoding a polypeptide with dehydroquinate dehydratase/shikimate dehydrogenase activity can confer resistance to inhibitors. The expression of such a gene leads to a herbicide-resistant plant, as has been shown for a further chorismate biosynthesis enzyme, namely enolpyruvylshikimate-3-phosphate synthase.

In other words, providing the herbicide target furthermore makes possible a method for identifying a dehydroquinate dehydratase/shikimate dehydrogenase which are not inhibited by the inhibitors according to the invention. An enzyme which differs thus from the dehydroquinate dehydratase/shikimate dehydrogenase according to the invention is referred to hereinbelow as a dehydroquinate dehydratase/shikimate dehydrogenase variant. The abovementioned method is also a subject of the present invention.

In a preferred embodiment, the abovementioned method for generating variants of the nucleic acid sequence SEQ ID No. 1 or SEQ ID No. 3 consists of the following steps:

a) expression of the proteins encoded by SEQ ID No. 1 or SEQ ID No. 3 in a heterologous system or in a cell-free system;

b) random or directed mutagenesis of the protein by modification of the nucleic acid;

c) measuring the interaction of the modified gene product with the herbicide;

d) identification of derivatives of the protein which interact less;

e) assaying the biological activity of the protein following application of the herbicide;

f) selection of the nucleic acid sequences which display a modified biological activity toward the herbicide.

The sequences selected by the above-described method are advantageously introduced into an organism. Accordingly, the invention furthermore relates to an organism generated by this method; the organism is preferably a plant.

Then, intact plants are regenerated, and the resistance to the herbicide is verified in intact plants.

Modified proteins and/or nucleic acids capable of conferring, in plants, resistance to herbicides can also be generated from the sequence SEQ ID No. 1 or SEQ ID No. 3 via what is known as site-directed mutagenesis; for example the stability and/or enzymatic activity of enzymes, or properties such as binding of the abovementioned inhibitors according to the invention, can be improved or modified in a highly targeted fashion using this mutagenesis.

For example, a site-directed mutagenesis method in plants, which can be used advantageously, has been described by Zhu et al. (Nature Biotech., Vol. 18, May 2000: 555-558).

Moreover, modifications can be achieved via the PCR method described by Spee et al. (Nucleic Acids Research, Vol. 21, No. 3, 1993: 777-78) using dITP for random mutagenesis or by the method further improved by Rellos et al. (Protein Expr. Purif., 5, 1994: 270-277).

Another possibility of generating these modified proteins and/or nucleic acids is an in-vitro recombination technique for molecular evolution which has been described by Stemmer et al. (Proc. Natl. Acad. Sci. USA, Vol. 91, 1994: 10747-10751), or the combination of the PCR and recombination methods described by Moore et al. (Nature Biotechnology Vol. 14, 1996: 458-467). Another route for the mutagenesis of proteins is described by Greener et al. in Methods in Molecular Biology (Vol. 57, 1996: 375-385). EP-A-0 909 821 describes a method for modifying proteins using the microorganism E. coli XL-1 Red. During replication, this microorganism generates mutations in the nucleic acids introduced, and thus leads to a modification of the genetic information. Advantageous nucleic acids and the proteins encoded by them can be identified readily via isolating the modified nucleic acids or the modified proteins and carrying out resistance tests. After their introduction into plants, they are capable of manifesting resistance therein and thus lead to resistance to the herbicides.

Further mutagenesis and selection methods are, for example, methods like the in-vivo mutagenesis of seeds or pollen and the selection of resistant alleles in the presence of the inhibitors according to the invention, followed by genetic and molecular identification of the modified, resistant allele; furthermore, mutagenesis and selection of resistances in tissue culture by multiplying the culture in the presence of successively increasing concentrations of the inhibitors according to the invention. The increase in the spontaneous mutation rate by means of chemical/physical mutagenic treatment can be exploited in the process. As described above, modified genes may also be isolated using microorganisms which show endogenous or recombinant activity of the proteins encoded by the nucleic acids used in the method according to the invention and which are sensitive to the inhibitors identified in accordance with the invention. Growing microorganisms on media with increasing concentrations of inhibitors according to the invention permits the selection and evolution of resistant variants of the targets according to the invention. The mutation frequency, in turn, can be increased by mutagenic treatments.

In addition, methods are available for the targeted modification of nucleic acids (Zhu et al. Proc. Natl. Acad. Sci. USA, Vol. 96, 8768-8773 and Beethem et al., Proc. Natl. Acad. Sci. USA, Vol 96, 8774-8778).

These methods make it possible to replace, in the proteins, those amino acids which are important for binding inhibitors by amino acids which are functionally equivalent, but which prevent binding of the inhibitor.

The invention furthermore relates to a method for generating nucleotide sequences which encode gene products with a modified biological activity, the biological activity being modified in such a way that it is increased. Increased activity is understood as meaning an activity which, in comparison with the original organism or the original gene product, is at least 10% higher, preferably at least 30% higher, especially preferably at least 50% higher, very especially preferably at least 100% higher. Moreover, the biological activity can have been modified in such a way that the substances and/or compositions according to the invention no longer bind, or no longer bind correctly, to the nucleic acid sequences and/or the gene products encoded by them. No longer or no longer correctly is understood as meaning, for the purposes of the invention, that the substances bind at least 30% less, preferably at least 50% less, especially preferably at least 70% less, very especially preferably at least 80% less or no longer at all to the modified nucleic acids and/or gene products in comparison with the original gene product or the original nucleic acids.

Yet another aspect of the invention thus relates to a transgenic plant which has been genetically modified by the above-described method according to the invention.

Genetically modified transgenic plants which are resistant to the substances found by the methods according to the invention and/or to compositions comprising these substances may also be generated by overexpressing the nucleic acids SEQ ID No. 1 or SEQ ID No. 3 used in the methods according to the invention. The invention therefore furthermore relates to a method for generating transgenic plants which are resistant to substances found by a method according to the invention, which comprises the overexpression, in these plants, of nucleic acids with the sequence SEQ ID No. 1 or SEQ ID No. 3. A similar method is described by way of example in Lermantova et al., Plant Physiol., 122, 2000: 75-83.

The above-described methods according to the invention for generating resistant plants make possible the development of novel herbicides whose activity is as comprehensive as possible and independent of the plant species (so-called nonselective herbicides) in combination with the development of crop plants which are resistant to the nonselective herbicide. Crop plants which are resistant to nonselective herbicides have already been described on several occasions. In this context, the principles for generating resistance can be classified into:

a) the generation of resistance in a plant via mutation methods or recombinant methods by significantly overproducing the protein which acts as target for the herbicide and by, owing to the large excess of the protein which acts as target for the herbicide, the function performed by this protein in the cell being retained even after application of the herbicide.

b) The modification of the plant in such a way that a modified version of the protein acting as target for the herbicide is introduced and that the function of the newly introduced modified protein is not adversely affected by the herbicide.

c) The modification of the plant in such a way that a novel protein/RNA is introduced, wherein the chemical structure of the protein or of the nucleic acid, such as the RNA or the DNA, which is responsible for the herbicidal action of the low-molecular-weight substance, is modified in such a way that the modified structure prevents a herbicidal action from being developed, that is to say that the herbicide can no longer interact with the target.

d) The function of the target is replaced by a novel gene which is introduced into the plant, thus creating what is known as an alternative pathway.

e) The function of the target is taken over by another gene present in the plant, or its gene product.

The present invention therefore furthermore comprises the use of plants, the genes affected by the insertion of the T-DNA, with the nucleic acid sequences SEQ ID No. 1 or SEQ ID NO. 3, for the development of novel herbicides. The skilled worker is familiar with alternative methods for identifying the homologous nucleic acids, for example in other plants, using similar sequences such as, for example, using transposons. The present invention therefore also relates to the use of alternative insertion mutagenesis methods for inserting foreign nucleic acid into the nucleic acid sequence SEQ ID No. 1 or SEQ ID No. 3, into sequences derived from these sequences owing to the genetic code, and/or into their derivatives in other plants.

A further variant of the method for identifying polypeptides with dehydroquinate dehydratase/shikimate dehydrogenase activity which are resistant to the inhibitors according to the invention is based on the fact that the dehydroquinate dehydratase/shikimate dehydrogenase pathway is found not only in plants, but also in bacteria and fungi. Some of these microorganisms might comprise dehydroquinate dehydratase/shikimate dehydrogenase variants.

The method according to the invention for the targeted detection of said dehydroquinate dehydratase/shikimate dehydrogenase variants is based on incubating an organism with an inhibitor identified by the method according to the invention. If no growth inhibition, or only partial growth inhibition is observed, the dehydroquinate dehydratase/shikimate dehydrogenase is isolated from said organism and characterized with regard to its nucleic acid sequence. Partial growth inhibition is understood as meaning that the growth is reduced by only 50%, preferably 45%, especially preferably 20%, in comparison to a nonincubated organism. If appropriate, an existing resistance is potentiated by further mutations. In this context, the above-described mutagenesis methods may be employed.

In this context, any organism which contains enzymes of the shikimate pathway may be used. Especially preferred in this context are bacteria, plants and fungi.

The invention furthermore relates to transgenic organisms, preferably plants, whose propagation material and whose plant cells, plant tissues or plant parts, transformed with an expression cassette comprising the sequence of a dehydroquinate dehydratase/shikimate dehydrogenase variant which is not inhibited by the inhibitors according to the invention. The expression cassette is identical with the above-described embodiments of an expression cassette for the expression of dehydroquinate dehydratase/shikimate dehydrogenase, except that it contains said dehydroquinate dehydratase/shikimate dehydrogenase variant instead of the nucleic acid sequence of the dehydroquinate dehydratase/shikimate dehydrogenase.

The transgenic plants are generated with one of the above-described embodiments of the expression cassette according to the invention by customary transformation methods which have likewise been described above.

The expression efficacy of the recombinantly expressed dehydroquinate dehydratase/shikimate dehydrogenase gene can be determined for example in vitro by shoot-meristem propagation or by a germination test. Moreover, expression of the dehydroquinate dehydratase/shikimate dehydrogenase gene which has been modified with regard to type and level, and its effect on the resistance to dehydroquinate dehydratase/shikimate dehydrogenase inhibitors, can be tested in greenhouse experiments, using test plants.

The invention furthermore relates to the use of an expression cassette according to the invention for transforming plants, plant cells, plant tissues or plant parts. The preferred aim of the use is an increase in the dehydroquinate dehydratase/shikimate dehydrogenase content, or the content of a polypeptide with dehydroquinate dehydratase/shikimate dehydrogenase activity, in the plant. The transgenic plants are generated as described above via the transformation of a plant with at least one expression cassette according to the invention or at least one vector according to the invention. However, increased expression may also be achieved by the targeted mutagenesis of the promoter region of the natural dehydroquinate dehydratase/shikimate dehydrogenase gene in question.

Thus, an increased resistance to the dehydroquinate dehydratase/shikimate dehydrogenase inhibitors according to the invention can be achieved by overexpressing the gene sequence SEQ ID No. 1 or SEQ ID No. 3, which encodes a dehydroquinate dehydratase/shikimate dehydrogenase, or their functional equivalents. The transgenic plants thus generated are likewise subject matter of the invention.

The further embodiments of the invention which follow are likewise based on overexpressing dehydroquinate dehydratase/shikimate dehydrogenase. In addition to the abovementioned methodology, the overexpression of dehydroquinate dehydratase/shikimate dehydrogenase may be conferred by means of an expression cassette according to the invention or a vector according to the invention, each of which comprises one of the above-described nucleic acid sequences encoding a polypeptide with an increased dehydroquinate dehydratase/shikimate dehydrogenase activity. An increased activity is understood as meaning, in this context, an activity which is at least 10% higher, preferably at least 30% higher, especially preferably at least 50% higher, very especially preferably at least 100% higher than the dehydroquinate dehydratase/shikimate dehydrogenase encoded by SEQ ID No. 1 or SEQ ID No. 2.

By overexpressing dehydroquinate dehydratase/shikimate dehydrogenase, it is also possible to increase the dry matter content of a plant via increasing chorismate and the aromatic amino acids. This leads to an increased dry matter and increases the overall yield of the plants.

Moreover, overexpressing dehydroquinate dehydratase/shikimate dehydrogenase can increase the biosynthesis of the aromatic amino acids phenylalanine, tyrosine and tryptophan.

Plants which are preferably to be used in this context are crop plants such as cereals, maize, soybean, rice, cotton, sugarbeet, canola, sunflower, flax, hemp, potato, tobacco, tomato, oilseed rape, alfalfa, lettuce and the various tree, nut and grapevine species, and legumes.

Depending on the choice of promoter, expression may take place specifically in the leaves, in the seeds or other parts of the plant. Such transgenic plants, their propagation material and their plant cells, plant tissue or plant parts are a further subject matter of the present invention.

The invention will now be illustrated by the examples which follow, without being limited thereto.

Genetic engineering methods on which the use examples are based:

General Cloning Methods

Cloning methods such as, for example, restriction cleavages, DNA isolation, agarose gel electrophoresis, purification of DNA fragments, transfer of nucleic acids to nitrocellulose and nylon membranes, linking DNA fragments, transformation of E. coli cells, bacterial cultures and sequence analysis of recombinant DNA were carried out as described by Sambrook et al., Cold Spring Harbor Laboratory Press (1989); ISBN 0-87969-309-6. The transformation of Agrobacterium tumefaciens was carried out by the method of Hofgen and Willmitzer (Nucl. Acid Res. 16, (1988) 9877). The agrobacteria were grown in YEB medium (Veryliet et al., Gen. Virol. 26 (1975), 33).

The bacterial strains used hereinbelow (E. coli, XL-I Blue) were obtained from Stratagene or Qiagen. The agrobacterial strain used for the transformation of plants (Agrobacterium tumefaciens, C58C1 carrying Plasmid pGV2260 or pGV3850kan) was described by Deblaere et al. in Nucl. Acids Res. 13 (1985), 4777. As an alternative, the agrobacterial strain LBA4404 (Clontech) or other suitable strains may also be employed. Vectors which may be used for cloning are pUC19 (Yanish-Perron, Gene 33 (1985), 103-119) pbluescript SK− (Stratagene), pGEM-T (Promega), pZerO (Invitrogen), pBinl9 (Bevan et al., Nucl. Acids Res. 12 (1984), 8711-8720) and pBinAR (Hofgen and Willmitzer, Plant Science 66 (1990), 221-230).

Sequence Analysis of Recombinant DNA

Recombinant DNA molecules were sequenced using an ABI laser fluorescence DNA sequencer, using the method of Sanger (Sanger et al., Proc. Natl. Acad. Sci. USA 74 (1977), 5463-5467). Fragments resulting from a polymerase chain reaction were sequenced and verified to avoid polymerase errors in constructs to be expressed.

Unless otherwise specified, the chemicals used were obtained in analytical-grade quality from Fluka (Neu-Ulm), Merck (Darmstadt), Roth (Karlsruhe), Serva (Heidelberg) and Sigma (Deisenhofen). Solutions were made with conditioned, pyrogen-free water, termed H₂O in the text which follows, from a milli-Q Water System water conditioning system (Millipore, Eschborn). Restriction endonucleases, DNA-modifying enzymes and molecular biology kits were obtained from AGS (Heidelberg), Amersham (Braunschweig), Biometra (Gottingen), Roche (Mannheim), Genomed (Bad Oeynnhausen), New England Biolabs (Schwalbach/Taunus), Novagen (Madison, Wis., USA), Perkin-Elmer (Weiterstadt), Pharmacia (Freiburg) Qiagen (Hilden) and Stratagene (Heidelberg). Unless otherwise specified, they were used following the manufacturer's instructions.

### EXAMPLE 1

Cloning the Nicotiana tabacum Dehydroquinate Dehydratase/Shikimate Dehydrogenase Gene

Dehydroquinate dehydratase/shikimate dehydrogenase was cloned from tobacco flowers by the RT-PCR method. A sequence analysis confirmed that it was indeed tobacco dehydroquinate dehydratase/shikimate dehydrogenase. The following primers were used for this procedure:

| | |
|---|---|
| 5′DHD-BamHI: | AAG GAT CCG GAA GTT CGA TTG CAT AGC |
| 3′DHD-BamHI: | AAG GAT CCT TCT CTC GCT CGT TCA TAG G |

The PCR product is 1088 base pairs in size and was used for antisense and cosuppression inhibition of the dehydroquinate dehydratase/shikimate dehydrogenase gene.

To overexpress the protein, the full-length clone was amplified from tobacco flower DNA using the PCR method.

The following primers were used for this procedure:

5′ GGG GAG GCA ATG ACG AGG AAC GAA ACA CTA 3′
5′ ATT CCT CCG AAG CAC AAA TGG TAG GGC AGA 3′

This cDNA fragment, which is 1668 base pairs in length, contains an open reading frame of 1668 bases and encodes a protein of 556 amino acids. The transit peptide belonging to the pre-protein was not cloned by this procedure. Analyses of the polypeptide using the program GCG (Oxford Molecular) resulted in 100% identity of the nucleic acid and amino acid level with a Nicotiana tabacum protein described in the database (Accession Number: L 32794).

### EXAMPLE 2

Preparation of Dehydroquinate Dehydratase/Shikimate Dehydrogenase Antisense and Cosuppression Constructs

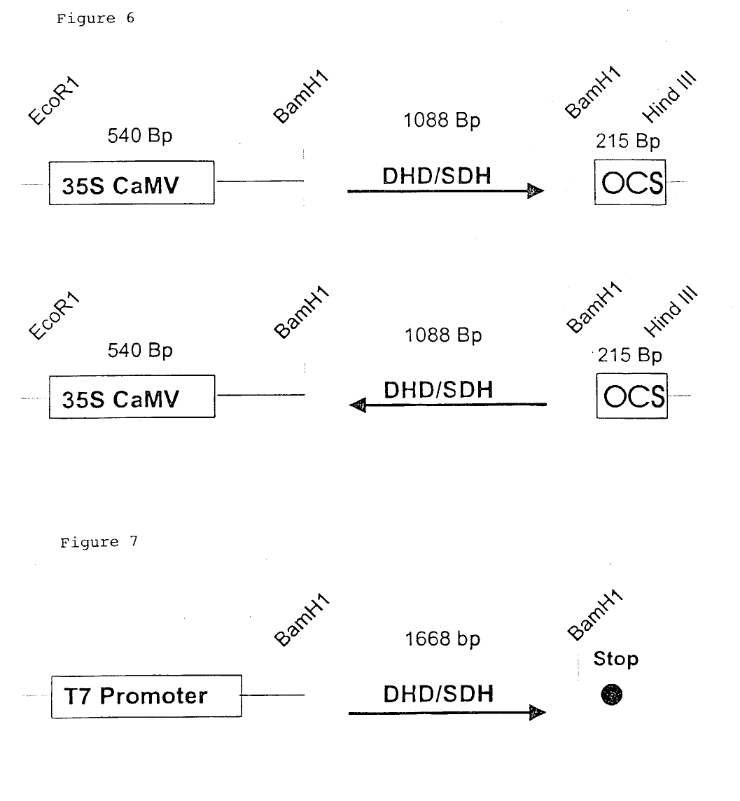

The 1088 base pair fragment of the Nicotiana tabacum dehydroquinate dehydratase/shikimate dehydrogenase was cloned into the binary vector pBinAR in sense orientation and in antisense orientation under the control of the 35S promoter, see FIG. 6. It was possible to use the BamHI cleavage sites dictated by the primers for cloning dehydroquinate dehydratase/shikimate dehydrogenase into the binary vector. The PCR product was cleaned using the Gene-Clean-Kit (Dianova GmbH, Hilden) and digested with BamHI. For ligation, vector pBin19AR was also cleaved with BamHI.

This construct was transferred into tobacco by agrobacterium-mediated transformation. Regenerated plants were tested for levels of dehydroquinate dehydratase/shikimate dehydrogenase mRNA. All antisense and sense plants tested whose dehydroquinate dehydratase/shikimate dehydrogenase mRNA levels were reduced exhibited an unambiguous phenotype. A strict correlation between phenotype and reduced mRNA level was found. Plants with a reduced dehydroquinate dehydratase/shikimate dehydrogenase mRNA exhibited mosaic leaves, reduced size—see FIGS. 2 to 4—and died during plant development.

### EXAMPLE 3

Generation of Transgenic Tobacco Plants

To generate transgenic tobacco plants (Nicotiana tabacum L. cv. Samsun NN), tobacco leaf discs were transformed with sequences of dehydroquinate dehydratase/shikimate dehydrogenase. To transform tobacco plants, 10 ml of an overnight culture of Agrobacterium tumefaciens which had been grown under selection conditions were spun down, the supernatent was discarded and the bacteria were resuspended in an equal volume of antibiotic-free medium. Leaf discs of sterile plants (approx. diameter: 1 cm) were bathed in this bacterial suspension in a sterile Petri dish. The leaf discs were subsequently plated onto MS medium (Murashige and Skoog, Physiol. Plant 15 (1962), 473) supplemented with 2% sucrose and 0.8% Bacto agar. After incubation for 2 days in the dark at 25 C, they were transferred to MS medium supplemented with 100 mg/l kanamycin, 500 mg/l claforan, 1 mg/l benzylaminopurine (BAP), 0.2 mg/l naphthylacetic acid (NAA), 1.6% glucose and 0.8% Bacto agar, and culturing was continued (16 hours light/8 hours dark). Growing shoots were transferred to hormone-free MS medium supplemented with 2% sucrose, 250 mg/l claforan and 0.8% Bacto agar.

### EXAMPLE 4

Analysis of Total RNA from Plant Tissues

Total RNA from plant tissue was isolated as described by Logemann et al., Anal. Biochem. 163 (1987), 21. For analysis, in each case 20 μg of RNA were separated in a formaldehyde-containing 1.5% agarose gel and transferred to nylon membranes (Hybond, mersham). The detection of specific transcripts was carried out s described by Amasino (Anal. Biochem. 152 (1986), 304). The DNA fragments employed as probe were radiolabeled with a Random Primed DNA Labeling Kit (Boehringer, Mannheim) and hybridized by standard methods (see Hybond Constructions, Amersham).

Hybridization signals were visualized by autoradiography using Kodak X-OMAT AR films.

FIG. 5 shows a Northern analysis of five tobacco plants (19-1, 19-4, 19-5, 83-2, 83-5) which have been transformed with a pBinAR antisense construct of DHD/SDH. As a control, the RNA of two wild-type plants is applied. DHD/SDH expression is reduced in the transgenic tobacco plants.

Wild-type and transgenic DHD/SDH plants are shown as a side view (FIG. 2) and from above (FIGS. 3 and 4). Severe growth inhibition in comparison with the wild type can be seen clearly (FIG. 2, wild type on the left). The reduced growth is correlated with a decreased DHD/SDH gene expression (FIGS. 5A and 5B). FIG. 5A shows Northern analyses of transgenic DHD/SHD plants of the Tl generation which exhibit greatly modified phenotypes. The analysis reveals that DHD/SHD gene expression is inhibited in plants with greatly modified phenotypes. FIG. 5B shows Northern analyses of transgenic DHD/SHD plants of the T1 generation with normal phenotype. The anaylsis of these plants reveals that no inhibition of DHD/SHD gene expression is observed in these plants even though a strong signal of the transferred fragments is present. To conclude, it can be said that a marked correlation between phenotype with reduced growth and inhibition of DHD/SHD gene expression is found.

### EXAMPLE 5

Detection of the Enzymatic Activity of Dehydroquinate Dehydratase/Shikimate Dehydrogenase

A. The shikimate dehydrogenase of the bifunctional dehydroquinate dehydratase/shikimate dehydrogenase enzyme catalyzes the following reaction:

shikimate+NADP [symbol] dehydroshikimate+NADPH

The formation of NADPH can be measured over 10 minutes at an OD of 334 nm. The reaction is started by adding 1 microliter of the extracted crude protein. The reaction buffer contains:

100 mM glycine-NaOH, pH: 9.9;

0.1 mM shikimate (Sigma);

0.1 mM NADP (AppliChem).

B. Addition of 3-dehydroshikimate allows the decrease of NADPH to be determined photometrically and thus the activity of 3-dehydroquinate dehydratase to be measured. This represents the back reaction from shikimate-3-dehydroquinate to give DHD/SHD.

C. Another enzyme assay of dehydroquinate dehydratase/shikimate dehydrogenase is carried out by measuring the two enzymes in a coupled back reaction:

3-dehydroquinate+NADP<=3-dehydroshikimate+NADPH<=shikimate+NADP

In this enzyme assay, the decrease in NADPH can be detected photometrically at an OD of 334. In this reaction, the enzymatic activity of both enzymes is detected in an assay.

### EXAMPLE 6

Cloning the Nicotiana tabacum Dehydroquinate Dehydratase/Shikimate Dehydrogenase into Expression Vectors of Heterologous Expression Systems

Suitable expression vectors are those for the expression of recombinant proteins in E. coli, but also baculovirus vectors for expressing dehydroquinate dehydratase/shikimate dehydrogenase in insect cells (Gibco BRL). Bacterial expression vectors are derived, for example, from pBR322 and carry a bacteriophage T7 promoter for expression. For expression, the plasmid is multiplied in an E. coli strain which carries an inducible gene for T7 polymerase (for example J1109(DE3); Promega). Expression of the recombinant protein is activated via the IPTG-mediated induction of T7 polymerase. If the recombinant protein is to be provided with a His tag for better purification by Ni-affinity chromatography, IPTG-inducible systems of Quiagen (pQE vectors) or Novagen (pET vectors) are the systems of choice. There are vectors with different reading frames, depending on the cleavage sites which are available.

The full-length dehydroquinate dehydratase/shikimate dehydrogenase gene was cloned into the pQE vector (FIG. 7) and transformed into E. coli. A single colony of this E. coli strain was incubated overnight at 37° C. in the growth medium “2xYT” (per liter: Bacto tryptone 16 g, yeast extract 10 g, NaCl 5 g, 50 mg/l ampicillin and 50 mg/l kanamycin). Next day, 50 ml of 2*YT were inoculated with 0.5 ml of the overnight culture and grown at 25° C. to an OD₆₀₀ of 0.6. Gene expression was induced by addition of IPTG (final concentration: 0.05 mM) and incubation was continued for 3 hours at 25° C. The cells were harvested at 4° C. by centrifugation for 10 minutes at 8000 rpm. The pellet was taken up in 3 ml of extraction buffer (50 mM NaH₂PO₄, 300 mM NaCl, 10 mM imidazole, 15% glycerol, 5 mM mercaptoethanol). The pellet was frozen in liquid nitrogen and again defrosted on ice. The cells were disrupted by sonication (4×45 seconds, 1 minute on ice). The cells were spun down for 20 minutes at 4° C. and 1500 rpm, and the supernatent was used directly for the enzyme measurements.

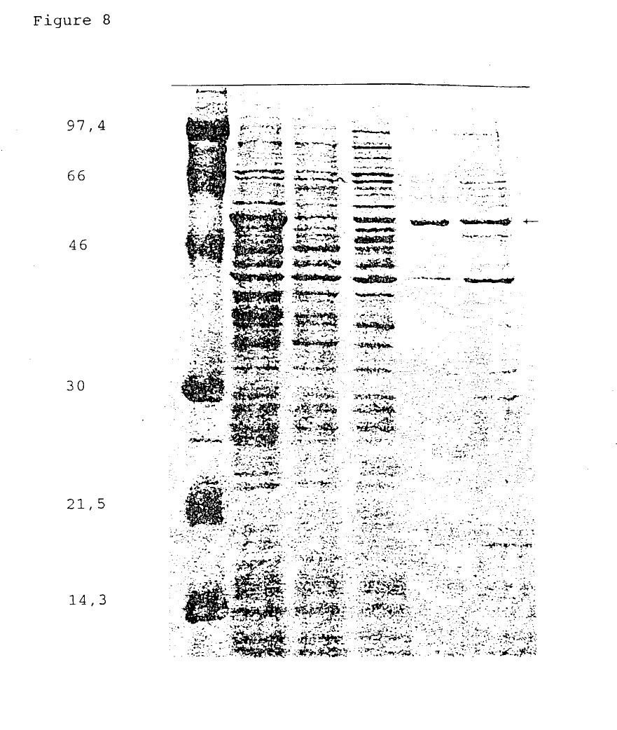

FIG. 8 shows the expressed DHD/SDH protein with a size of approx. 60 kD in the SDS-PAGE gel electrophoresis.

Lane 1 (left to right): protein marker, molecular weights top to bottom: 97.4 KD; 66 KD; 46 KD; 30 KD; 21.5 KD and 14.3 KD

Lane 2: induced DHD/SHD protein (crude extract, denatured) in the presence of 2 mM IPTG, 37° C. Molecular weight DHD/SHD: approx. 60 KD

Lane 3: uninduced control

Lane 4: induced DHD/SHD protein (crude extract, native) in the presence of 0.05 mM IPTG, 25° C.

Lane 5: induced DHD/SHD protein (purified on Ni-NTA material, native)

Lane 6: see Lane 5, but twice as much protein was applied

### USE EXAMPLE

Six substances whose IC50 value is in the μM range were identified in a comprehensive screening based on the activity assay described in Example 5A (see Table 1).

TABLE 1

| | | IC50 | | |
|---|---|---|---|---|
| No. | Structure | [μM] | Conc. | Effect |
| 1 | 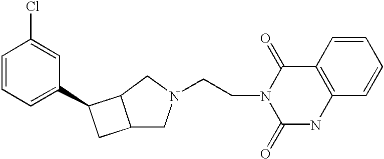 | 20 | | H |
| 2 | 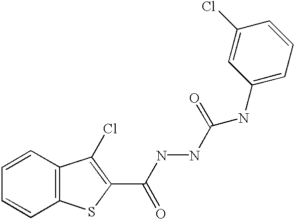 | 23 | | H |
| 3 | 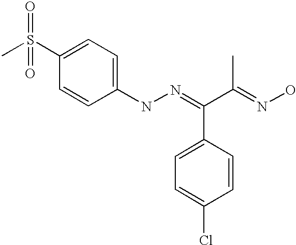 | 7 | | H |
| 4 | 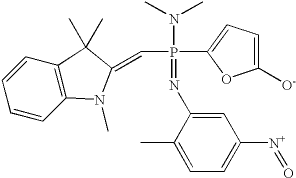 | 13 | | H |
| 5 | 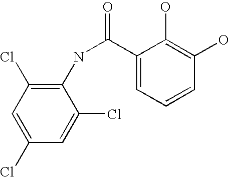 | 3 | | H |
| 6 | 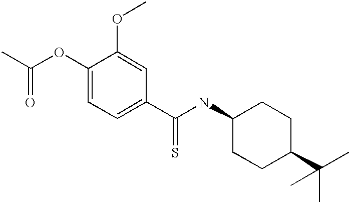 | 11 | | H |

The effect of the herbicidal compounds according to the invention on the growth of the duckweed Lemna paucicostatag is evident from the following test results:

Lemna minor was grown under nonsterile conditions in Petri dishes in 17 mmol/l MES buffer pH 5.5+1.5 mmol/l CaCl—2+1 g/l “Hakaphos spezial”.

To carry out the test, the Lemna cultures are washed and singled out into 0.5 ml of fresh nutrient solution in 48-well microtiter plates. The active ingredients are dissolved in DMSO at a concentration of 5 mmol/l and diluted 1:5 in water. 25 μl of this solution are used in the test.

The parameter measured is the fluorescence of the chlorophyll during the treatment. A herbicidal effect can be detected by comparison with an untreated control; it is identified in Table 1 by the symbol H.

We claim:

1. A method for identifying herbicidally active substances, which comprises influencing the transcription, expression, translation or the activity of the gene product of the amino acid sequence encoded by a nucleic acid sequence selected from the group consisting of
(a) a nucleic acid sequence with the sequences shown in SEQ ID NO: 1 or SEQ ID NO: 3, or
(b) a nucleic acid sequence which, owing to the degeneracy of the genetic code, can be deduced from the amino acid sequences shown in SEQ ID NO: 2 or SEQ ID NO: 4 by back translation, or
(c) functional analogs of the nucleic acid sequences shown in SEQ ID NO: 1 or SEQ ID NO: 3 which encode a polypeptide with the amino acid sequences shown in SEQ ID NO: 2 or SEQ ID NO: 4; or
d) functional analogs of the nucleic acid sequence [sic] shown in SEQ ID NO: 1 or SEQ ID NO: 3 which encode functional analogs of the amino acid sequences shown in SEQ ID NO: 2 or SEQ ID NO: 4; or
(e) nucleic acid sequence consisting of a part-regions [sic] of nucleic acid sequences a), b), c) or d) encoding a polypeptide with dehydroquinate dehydratase/shikimate dehydrogenase activity; or
(f) nucleic acid sequence consisting of at least 300 nucleotide units of nucleic acid sequences a), b), c) or d) encoding a polypeptide with dehydroquinate dehydratase/shikimate dehydrogenase activity;
and selecting the substances which reduce or block the transcription, expression, translation or the activity of the gene product in comparison with the gene product which has not been incubated with the substance.

2. A method as claimed in claim 1, which is carried out in an organism selected from the group of the bacteria, yeast, fungi or plants.

3. A method as claimed in any of claims 1 and 2, which is carried out in an organism selected from the group of the bacteria, yeast and fungi.

4. A method as claimed in claim 3, wherein an organism is used which is a conditional or natural mutant of sequence SEQ ID NO: 1 or SEQ ID NO: 3.

5. A method as claimed in claim 1, wherein a transgenic organism comprising
(a) a nucleic acid sequence with the sequence shown in SEQ ID NO: 1 or SEQ ID NO: 3, or
(b) a nucleic acid sequence which, owing to the degeneracy of the genetic code, can be deduced from the amino acid sequences shown in SEQ ID NO: 2 or SEQ ID NO: 4 by back translation, or
(c) functional analogs of the nucleic acid sequences shown in SEQ ID NO: 1 or SEQ ID NO: 3 which encode a polypeptide with the amino acid sequences shown in SEQ ID NO: 2 or SEQ ID NO: 4; or
(d) functional analogs of the nucleic acid sequence shown in SEQ ID NO: 1 or SEQ ID NO: 3 which encode functional analogs of the amino acid sequences shown in SEQ ID NO: 2 or SEQ ID NO: 4; or
(e) parts of the nucleic acid sequences a), b), c) or d) encoding a polypeptide with dehydroquinate dehydratase/shikimate dehydrogenase activity; or
(f) at least 300 nucleotide units of the nucleic acid sequences a), b), c) or d) encoding a polypeptide with dehydroquinate dehydratase/shikimate dehydrogenase activity;
or a vector comprising the abovementioned expression cassette is used, the transgenic organism being selected from the group consisting of bacteria, yeast, fungi, animal cells or plant cells.

6. A method as claimed in claim 5, wherein the transgenic organism is selected from the group consisting of bacteria and plant cells.

7. A method as claimed in any of claims 1, 2, 3, 4, 5 or 6, wherein
(a) the polypeptide is either expressed in enzymatically active form in a transgenic organism or an organism comprising the protein according to the invention is cultured;
(b) the polypeptide obtained in step a) is incubated with redox equivalents and with a chemical compound, either directly in the quiescent or growing organism, in the cell digest of the organism, in partially purified form or in homogeneously purified form;
(c) a chemical compound is selected by step b), which compound inhibits the polypeptide with dehydroquinate dehydratase/shikimate dehydrogenase activity; and
(d) after a suitable reaction time, the enzymatic activity of the polypeptide is determined in comparison with the activity of the uninhibited enzyme.

8. A method as claimed in claim 1, consisting of the following steps:
(a) generation of organisms which, following transformation with an expression cassette comprising
(a) a nucleic acid sequence with the sequence shown in SEQ ID NO: 1 or SEQ ID NO: 3, or
(b) a nucleic acid sequence which, owing to the degeneracy of the genetic code, can be deduced from the amino acid sequences shown in SEQ ID NO: 2 or SEQ ID NO: 4 by back translation, or
(c) functional analogs of the nucleic acid sequences shown in SEQ ID NO: 1 or SEQ ID NO: 3 which encode a polypeptide with the amino acid sequences shown in SEQ ID NO: 2 or SEQ ID NO: 4; or
(d) functional analogs of the nucleic acid sequence shown in SEQ ID NO: 1 or SEQ ID NO: 3 which encode functional analogs of the amino acid sequences shown in SEQ ID NO: 2 or SEQ ID NO: 4; or
(e) parts of the nucleic acid sequences a), b), c) or d) encoding a polypeptide with dehydroquinate dehydratase/shikimate dehydrogenase activity; or
(f) at least 300 nucleotide units of the nucleic acid sequences a), b), c) or d) encoding a polypeptide with dehydroquinate dehydratase/shikimate dehydrogenase activity;
or a vector comprising the abovementioned expression cassette is used, the transgenic organism being selected from the group consisting of bacteria, yeast, fungi, animal cells or plant cells;
comprise an additional DNA sequence encoding an enzyme with dehydroquinate dehydratase/shikimate dehydrogenase activity and which are capable of overexpressing an enzymatically active dehydroquinate dehydratase/shikimate dihydrogenase;
(b) applying a substance to the organism of claim a) [sic];
(c) determining the growth or the viability of the transgenic and of the untransformed organism after application of the chemical substance.

9. A method as claimed in claim 8, wherein the transgenic organisms employed are transgenic plants, plant cells, plant tissues or plant parts.

10. A method as claimed in any of claims 1-9, wherein the substances are identified in a high-throughput screening.

11. A method which comprises applying the substances identified by the method as claimed in any of claims 1 to 10 to a plant in order to assay the herbicidal activity and selecting those substances which demonstrate herbicidal activity.

12. A herbicide identified by methods claimed in any of claims 1 to 10.

13. The use of substances of claim 12 as herbicides or growth regulators.

14. A method for generating variants of the nucleic acid sequences SEQ ID NO: 1 or SEQ ID NO: 3, which comprises the following process steps:
(a) expression, in a heterologous system or in a cell-free system, of the proteins encoded by SEQ ID NO: 1 or SEQ ID NO: 3;
(b) randomized or directed mutagenesis of the protein by modification of the nucleic acid;
(c) measuring the interaction of the modified gene product with the herbicide;
(d) identification of derivatives of the protein and of the nucleic acid sequences encoding the protein which demonstrate a lesser degree of interaction;
(e) assaying the biological activity of the protein following application of the herbicide.

15. A method as claimed in claim 14, wherein the organism is a bacterium, a plant or a fungus.

16. A method of generating variants of the nucleic acid sequences SEQ ID NO: 1 or SEQ ID NO: 3, which comprises the following process steps:
(a) detection of organisms which are not, or only partially, inhibited by substances as claimed in claim 12;
(b) isolation and characterization of the for [sic] a polypeptide with dehydroquinate dehydratase/shikimate dehydrogenase activity from the organisms identified by step (a);
(c) if appropriate, optimization of the resistance by randomized or directed mutagenesis of the nucleic acid identified by (b).

17. A method as claimed in claim 16, wherein the organism is selected from the group of the bacteria and fungi.

18. A method for generating transgenic plants, plant tissues or plant cells which are resistant to substances found by a method as claimed in any of claims 1 to 10, wherein nucleic acids with the sequences SEQ ID NO: 1, SEQ ID NO: 3 or nucleic acid sequences of claims 14, 15, 16 or 17 are overexpressed in these plants.

19. A method for generating a transgenic plant, which comprises transforming, into a plant, an expression cassette encompassing
(a) a nucleic acid sequence with the sequence shown in SEQ ID NO: 1 or SEQ ID NO: 3, or
(b) a nucleic acid sequence which, owing to the degeneracy of the genetic code, can be deduced from the amino acid sequences shown in SEQ ID NO: 2 or SEQ ID NO: 4 by back translation, or
(c) functional analogs of the nucleic acid sequences shown in SEQ ID NO: 1 or SEQ ID NO: 3 which encode a polypeptide with the amino acid sequences shown in SEQ ID NO: 2 or SEQ ID NO: 4; or
(d) functional analogs of the nucleic acid sequence shown in SEQ ID NO: 1 or SEQ ID NO: 3 which encode functional analogs of the amino acid sequences shown in SEQ ID NO: 2 or SEQ ID NO: 4; or
(e) parts of the nucleic acid sequences a), b), c) or d) encoding a polypeptide with dehydroquinate dehydratase/shikimate dehydrogenase activity; or
(f) at least 300 nucleotide units of the nucleic acid sequences a), b), c) or d) encoding a polypeptide with dehydroquinate dehydratase/shikimate dehydrogenase activity;
or a vector comprising the abovementioned expression cassette.

20. A transgenic plant as claimed in claim 19 with an increased dry matter in comparison with a nontransgenic plant.

21. A transgenic plant of claim 19 with an increased amount of aromatic amino acids in comparison with a nontransgenic plant.

22. A method for controlling undesired vegetation, which comprises applying, to the undesired plants, a substance of claim 12.

23. The use of a substance as claimed in claim 12 for controlling undesired vegetation.
